(19) United States
(12) Patent Application Publication
(10) Pub. No.: US 2001/0049104 A1
(43) Pub. Date: Dec. 6, 2001

(54) Methods for modulating cellular and organismal phenotypes

(76) Inventors: Willem P.C. Stemmer, Los Gatos, CA (US); Jeremy Minshull, Menlo Park, CA (US); Robert J. Keenan, San Francisco, CA (US)

Correspondence Address: LAW OFFICES OF JONATHAN ALAN QUINE, P O BOX 458, ALAMEDA, CA, 94501

(21) Appl. No.: 09/817,015

(22) Filed: Mar. 23, 2001

Related U.S. Application Data
(60) Provisional application No. 60/191,782, filed on Mar. 24, 2000.
(60) Provisional application No. 60/262,617, filed on Jan. 17, 2001.

(51) Int. Cl. C12Q 1/68; C12N 15/85; C12N 15/63

(52) U.S. Cl. 435/6; 435/455

(57) ABSTRACT

Methods for identifying and controlling the genetic and metabolic pathways underlying complex phenotypes are provided. Conjoint polynucleotide segments that contribute to or disrupt elements of a multigenic phenotype are produced and expressed in cells of interest. Conjoint polynucleotide segments are recombined and/or mutated to give rise to libraries of recombinant concatamers which are expressed in cells of interest. Libraries of conjoint polynucleotide segments and recombinant concatamers are expressed episomally or integrated into the DNA of organelles or chromosomes. Cells are screened or selected to identify members of the population of cells exhibiting a desired phenotype. Libraries and vectors comprising conjoint polynucleotide segments and recombinant concatamers, as well as cells expressing such libraries and vectors or their components are provided. Kits containing conjoint polynucleotide segments, recombinant concatamers, vectors including such polynucleotides, and cells including such polynucleotides and vectors are provided.

### CROSS REFERENCE TO RELATED APPLICATIONS

This application claims priority to and benefit of U.S. Provisional Applications No. 60/191,782, filed Mar. 24, 2000, and No. 60/262,617, filed Jan. 17, 2001, the disclosures of which are incorporated herein in their entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Complex cellular and organismal phenotypes are typically controlled by cascades of regulators, including signaling pathways and effectors, such as transcription factors. Changes in activities of only one or a few of these regulators can cause dramatic but concerted alterations of phenotypes, for example in processes like sporulation of bacteria and slime molds, switches to hyphal growth in fungi, and sexual determination and differentiation and development in metazoans.

Signaling pathways contain a variety of elements that can control multiple downstream events (see, Madhani and Fink (1997) Science 275:1314-7). For example, in cell cycle control, the p34^{cdc2} kinase initiates chromosome condensation, nuclear envelope breakdown and spindle assembly by phosphorylation of substrates. Likewise, transcription factors often activate the expression of multiple genes required for a complex phenotype such as expression of all the correct genes in a certain tissue, or expression of all the catabolic genes (e.g., encoding enzymes, etc.) required to metabolize a certain substrate.

Variations in such master control genes results in complex downstream alterations, frequently resulting in complex phenotypic changes. For example one or a few mutants in a homeotic gene may lead, e.g., to the antenna of a fruit fly being transformed into a leg, a process which has been impossible to achieve by concerted mutation of all of the genes normally responsible for leg development. However, frequently the result of altering the sequence, expression or regulation of a master control gene is deleterious, sometimes in foreseen ways, but often in an unpredictable manner.

The present invention provides methods for identifying and evolving cellular and organismal phenotypes, for example, the complex pathways, including master regulators and molecular switches, as well as the myriad cellular targets that result in a phenotype of interest, making it possible to control complex phenotypes with desired results.

In addition, the present invention provides methods and compositions for concerted modification of any peptide or active nucleic acid element, including both phenotype modifiers and, e.g., enzymatic modulators. These and further features of the invention are provided by review of the following.

### SUMMARY OF THE INVENTION

The present invention provides methods for identifying and controlling genetic elements underlying cellular and organismal phenotypes, including complex phenotypes. The complex phenotype can be the product of one or more elements of a metabolic or genetic pathway, or of multiple related or unrelated metabolic or genetic pathways. Phenotypes produced through the action or influence of a cellular target, such as enzymes, transcription factors, receptors, hormones, and the like, are amenable to regulation by modulating, e.g., enhancing or inhibiting, activity or expression of a known or unknown target. In addition, phenotypes that are the product of the combined activity of multiple genes or proteins (targets) can be modulated by the methods provided herein. For example, multigenic phenotypes such as cell cycle state, cell cycle progression, cell morphology, DNA replication activity, transcriptional activity, nucleic acid recombination activity, meiosis, timing of secondary metabolite production, quantity of secondary metabolite production, oil content and composition, fat content and composition, sugar content and composition, starch content and composition, protein content and composition, phytochemical content and composition, nutraceutical content and composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity, are subject to manipulation and improvement by the methods of the present invention.

Multiple genetic elements which can contribute to or which can modulate, e.g., a complex phenotype are joined together in the form of conjoint polynucleotide segments and used to identify and manipulate one or more elements or components of the metabolic and genetic pathways that control a phenotype of interest. Conjoint polynucleotide segments of the invention can be, e.g., DNA, RNA, or other coding materials, including genomic DNA, cDNA, sense-strand DNA, antisense DNA, DNA encoding a dominant negative protein variant or a transdominant protein or peptide variant, DNA encoding a peptide modulator, DNA encoding a peptide having from about 5 to about 100 amino acids, DNA or RNA encoding a molecular decoy, viral DNA or RNA, sense-strand RNA, antisense RNA, tRNA, ribozymes, RNPs and RNA components of the splicing machinery. The segments can be elements of a single metabolic or genetic pathway or of multiple metabolic or genetic pathways.

In one embodiment, a library of expressible polynucleotide sequences that include conjoint polynucleotide segments that are candidates for altering expression or activity of one or more components of an endogenous pathway are introduced into a population of cells or intracellular organelles. In some embodiments, conjoint polynucleotide segments that are candidates for altering one, two or more (i.e., multiple) components or elements of an endogenous multigenic pathway are introduced. The cells are then screened for a desired alteration in their phenotype, e.g., modulation of a cellular target.

In another embodiment, a population of conjoint polynucleotide segments that contribute to or disrupt elements of a multigenic phenotype are recombined or mutated to generate a library of recombinant or variant concatamers. Optionally the mutation or recombination processes are performed recursively. In some cases, additional diversity generating techniques are performed in conjunction with the recombination process. The concatamers are introduced into recipient cells, or intracellular organelles, and the cells are screened for a desired effect on a phenotype. In some cases, multiple conjoint polynucleotide segments are introduced into cells in a combinatorial fashion. Combinations can include different combinations of “supersets” or combinations of subsets of the same “superset” on different episomes. In some cases, the recombinant concatamers are integrated into a chromosome or into the DNA of an intracellular organelle such as a chloroplast or mitochondria. Recipient cells include bacterial cells, yeast cells, fungal cells, plant cells and animal cells.

In alternative embodiments, libraries of nucleic acids including one or more polynucleotide segment under the control of transcriptional regulatory sequences are introduced into populations of cells, such that subsets of two or more library members are introduced into individual cells where they alter the expression or activity of one or more components of a multigenic pathway to produce desired phenotypes. Optionally, one or more members of the library are identified or recovered from the cells with desired phenotypes. The recovered library members can be recombined and/or mutated, optionally recursively, to generate recombinant polynucleotide segments, which can, in turn, be introduced into host cells and selected for their ability to modulate or produce a desired phenotype. In an embodiment, the introduced recombinant polynucleotide segment is integrated into a chromosome. Optionally, host cells are regenerated to produce a multicellular transgenic organism.

Individual polynucleotide segments are, alternatively, random or pre-selected by any one of a variety of means. For example, members of the library of conjoint polynucleotide segments can be pre-selected by introducing the library into recipient cells, selecting cells with a desired phenotype, and recovering the nucleic acid comprising the conjoint polynucleotide segments from the selected cell. Alternatively, methods including computational analysis (e.g., genomics, comparative genomics), expression analysis, screening encoded peptides or activities, yeast two-hybrid analysis, flow cytometry, metabolic modeling and/or flux analysis are used to pre-select polynucleotide segments.

Many phenotypes, including multigenic phenotypes, are typically regulated by many interacting factors, including transcription factors, molecular switches, promoter and enhancer effects, and the like, which act at the transcriptional, post-transcriptional and translational or post-translational level. In some embodiments, the phenotype is controlled by an epigenetic mechanism. As that term is used herein, epigenetic mechanisms include: e.g., chromatin silencing, methylation, maternal effects, antisense suppression, sense suppression, cosuppression, promoter alteration, homology-dependent mechanisms, aminoacylation, post-transcriptional gene silencing, post-translational gene silencing, DNA recombination, and the like.

In some embodiments, the conjoint polynucleotide segments are present in a vector, such as an episomal vector. Such vectors include plasmids, viruses, pro-viruses, artificial chromosomes (e.g., BACs, YACs, etc.), transposons, bacteriophages, and phagemids. Optionally, the episomal vector is integrated into a chromosome of a recipient cell or organism, or into the DNA of a intracellular organelle. Such episomal vectors are a feature of the invention.

In some embodiments, one or more recombinant concatamers are recovered from a cell with a desired phenotype and optionally introduced (with or without further modification) into a host cell to produce a transgenic organism. In some embodiments, one or more genetic elements corresponding to subsequences of the conjoint polynucleotide segments or recombinant concatamers are isolated, and optionally, further recombined and/or mutated to generate a set of isolated gene homologues which can be selected for a desired property.

In other embodiments, methods for modulating the activity of cellular targets are provided. Members of a library of polynucleotides encoding pre-selected peptides, e.g., peptide modulators, are joined to generate a population of conjoint polynucleotide segments operably linked to a transcription regulatory sequence. The conjoint polynucleotide segments are expressed in vitro or in vivo to produce a multipeptide including multiple discrete peptide segments, optionally joined by linker sequences, e.g., linkers subject to proteolytic cleavage. Then, one or more conjoint polynucleotide segments encoding a multipeptide with at least one peptide capable of modulating activity of a target are identified. Optionally, the identified conjoint polynucleotide segments are recombined or mutated, one or more times, e.g., recursively, to produce a library of recombinant concatamers. The recombinant concatamers are expressed, and recombinant concatamers with desired properties are identified. The pre-selected peptide sequences can be either the same or different amino acid sequences, and can possess identical, similar or different activities. Typically, the individual peptide components range in length from about 5 to about 500 amino acids, more typically from about 5 to about 150 amino acids, most typically from about 5 to about 100, often from about 5 to about 50 amino acids. In some embodiments, the peptides are peptide modulators, such as peptide inhibitors, of an enzyme or class of enzymes.

Accordingly, targets, such as one or more enzyme, or a class of enzymes, e.g., proteases, hydrolases, lipases, esterases, or amylases are modulated by the peptide modulators of the invention. For example, such targets can be intracellular molecules, extracellular molecules or cell surface molecules. Modulators can affect one or more of target binding to a substrate, catalytic activity, anabolic activity, stability, substrate specificity, function in selected environments, and the like. In some cases multiple targets that are at least two different enzymes are modulated by one, or more than one, of the components of a multipeptide. In some cases the targets are multiple members of a class of related enzymes.

The polynucleotide segments can be generated by such methods as a polymerase chain reaction or by producing synthetic oligonucleotides. For example the synthetic oligonucleotides can be random, partially randomized, or designed oligonucleotides, e.g., N-mers. The library of pre-selected peptides with desired properties can be produced by a variety of methods, including well-known screening procedures and consideration of statistical or structural information relative to one or more target of interest. In some embodiments, the peptides are pre-selected by expressing them in cells, and selecting cells with a desired phenotype. For example, a library of pre-selected peptides can be assembled by expressing fusion proteins capable of displaying one or more variable peptide moiety in vitro, e.g., by ribosomal display, or on the surface of a cell or phage, e.g., by expression on the surface of a bacterial or yeast cell as a fusion to cell surface protein, such as OmpA. The displayed fusions are screened, e.g., using a labeled target, such as a model enzyme, to identify variable peptide moieties with desired properties. These variable peptide moieties with desired properties then contribute to a library of pre-selected peptides. In this manner, libraries in excess of about 100, 1000, 10,000, 100,000, or 1,000,000 can be produced. The polynucleotide segments encoding these pre-selected peptides can then be joined to produce conjoint polynucleotide segments.

Although generally described in terms of in vivo expression and screening, in vitro expression and screening approaches can also be used. For example, an in vitro transcription and/or translation system can be used to produce any conjoint polynucleotide segments or polypeptides (or multipeptides) of the invention, which can be screened by any available method.

Libraries of conjoint polynucleotide segments, recombinant concatamers and vectors comprising such polynucleotide sequences are an aspect of the invention. Such libraries typically comprise DNA, including, e.g., genomic DNA, cDNA, sense-strand DNA, antisense DNA, DNA encoding a dominant negative protein variant, and DNA encoding a transdominant protein variant, or can comprise RNA, including, e.g., sense-strand RNA, antisense RNA, tRNA, ribozymes, RNPs and RNA components of the splicing machinery. The DNA and RNA nucleic acids can comprise all or part of a promoter, enhancer, or structural gene, including e.g., transcription factors, e.g., zinc finger proteins, enzymes, receptors, hormones, and signaling peptides or polypeptides, or combinations thereof.

In some embodiments, the selected or evolved conjoint polynucleotide segments (e.g., recombinant or variant concatamers) are recovered and introduced into a cell or organism to produce a transgenic cell or organism having a desired phenotype. Cells and organisms produced by the methods of the invention are an aspect of the invention.

Kits containing polynucleotides, vectors, libraries and/or cells including such polynucleotides, vectors or libraries, are also an aspect of the invention.

### BRIEF DESCRIPTION OF THE FIGURES

FIG. 1 is a schematic illustration showing the correspondence of multiple genetic elements that make up an episomal vector comprising conjoint polynucleotide segments with multiple genes of a genetic or metabolic pathway.

FIG. 2 is a schematic illustration showing the combinatorial arrangement of elements that make up an episomal vector comprising conjoint polynucleotide segments.

FIG. 3 is a schematic illustration showing the diversification of an episomal vector comprising conjoint polynucleotide segments to produce a set recombinant or variant concatamers which influence multiple components of a genetic or metabolic pathway.

FIG. 4 is a schematic illustration showing the recovery of optimized elements, and their use in the isolation and evolution of individual genes underlying a complex phenotype.

FIG. 5 is a schematic illustration of cellular transdifferentiation induced by a recombinant concatamer.

FIG. 6 is a schematic tabulation of a multivariant analysis correlating transdifferentiation with combinations of genetic elements.

### DETAILED DISCUSSION OF THE INVENTION

Episomes, including plasmids and viruses, can be rapidly evolved at a rate much greater than that of genomic evolution. The present invention takes advantage of the rapid rate of episomal evolution and applies it to the regulation of cellular and organismal phenotypes, including complex, multigenic phenotypes. For example, by spatially combining sequences that are related functionally, such as members of a metabolic pathway or genetic pathway, or of related metabolic or genetic pathways, or of different genes or pathways that interact to control a phenotype or group of phenotypes, the invention provides for the rapid evolution of phenotypes that are otherwise not readily accessible to genetic manipulation due to the complexity of the component genetic elements, or to their disconcerted control mechanisms or spatial separation. The methods of the invention are suitable for modifying phenotypes controlled by multiple known, or unknown genetic elements, including such disparate components as enzymes, transcription factors, receptors, and hormones, among others.

Multiple methods for regulating metabolic and genetic pathways are known. In general, these methods involve modulating, e.g., enhancing or repressing, individual or multiple elements of the pathway. While generally effective for regulating phenotypes due to single known genes, prior methods that result in the mutation of structural or regulatory components of the pathway, can be applied only with difficulty to multiple elements simultaneously.

For example, a relevant pathway can be regulated by extracellular factors, such as hormones or compounds in the environment, inducing a transcription factor which increases transcription of several key metabolic enzymes. Controlling the pathway, and hence, controlling the phenotype, can be performed, and in some cases is necessarily performed at several levels, e.g., binding of the hormone, expression of the transcription factor, binding of the transcription factor to promoter/enhancers sequences, competing factors, post-transcriptional processing such as splicing, etc. The present invention provides methods for rapidly identifying and evolving regulators that modulate, individually or simultaneously, one or more target contributing to a phenotype of interest. In addition to such regulators as dominant-negative, transdominant and peptide modulators, the present invention also uses epigenetic means, such as antisense, and/or sense suppression at a post-transcriptional level, to regulate multiple aspects of the pathway.

Definitions

Unless defined otherwise, all technical and scientific terms used herein have the meaning commonly understood by a person skilled in the art to which this invention pertains.

A “multigenic phenotype” refers to a phenotype that is the result of multiple gene products. Such products can be encoded by quantitative trait loci, and/or by genes which encode members of a single metabolic or genetic pathway, or of several related or unrelated metabolic or genetic pathways.

Gene products belong to the same “metabolic pathway” if they, in parallel or in series, act on the same substrate, produce the same product, or act on or produce a metabolic intermediate between the same substrate and product. Similarly, gene products belong to the same “genetic pathway” if they, in parallel or in series, directly or indirectly, regulate the same gene, or are regulated by the same gene product. Similarly, gene products belong to the same “phenotypic pathway” if they, in parallel or in series, contribute to the same phenotype.

An “epigenetic” phenomenon in classical parlance was often used to refer to a cytoplasmically directed form of regulation, such as a maternal effect. The term is also used to refer to paragenetic alterations in the genome of an organism, such as alterations which result from a mechanism other than alteration of the sequence of the gene (e.g., chromatin conformation, methylation, etc.). In the present invention, the term optionally refers to either of these phenomena (depending on context), and, also can refer to regulation by episomally encoded regulators of gene activity such as episomally encoded anti-sense sequences, sense sequences, ribozymes, nucleic acids encoding transdominant proteins, nucleic acids encoding peptide modulators, molecular decoys and the like.

The term “conjoint polynucleotide segments” refers to multiple polynucleotide segments that are joined together in a linear, end-to-end, array. The segments can be like or unlike polynucleotide sequences, and can be arrayed head-to-head, tail-to-tail, head-to-tail, (i.e., sense-to-sense, antisense-to-antisense, or sense-to-antisense) or any combination thereof. The segments so joined can be “random,” that is, not identified or selected based on any pre-determined criteria from a library or pool of polynucleotide segments. Alternatively, the segments can be “pre-selected” based on predetermined structural, e.g., sequence related, or functional criteria. The term is applied exclusively to denote an assembly of unit segments, wherein each unit typically maintains structural and/or functional integrity distinct from other component segments of the polynucleotide, and/or encoded polypeptide (multipeptide). To distinguish this characteristic, the term “multipeptide” is used to refer to a polypeptide encoding multiple, typically short, functionally and structurally distinct peptide sequences linked together in a single translation product. It should be noted that the term “conjoint polynucleotide segments” does not denote a nucleic acid encoding a single functional protein, such as a fusion protein, pro-or pre-pro-polypeptide or peptide, wherein the assembly encodes a single polypeptide with an integral structure and/or function. This does not foreclose the possibility that fortuitous additive effects between components of a multipeptide will result in the production of a novel functional unit.

“Recombinant concatamers” or “variant concatamers” are conjoint polynucleotide segments that are the product of one or more diversification, e.g., mutation and/or recombination, process, e.g., a DNA shuffling process.

The tern “pre-selected” when referring to a library, a polynucleotide segment, or other nucleic acid, or an encoded product such as a peptide, indicates that the molecule (nucleic acid, or encoded product) or library meets one or more defined criteria, e.g., relating to sequence, structural, or functional characteristics of the molecule or library.

A “library” of polynucleotide sequences is a collection of different polynucleotide sequences that share a common structural, functional, or other characteristic, e.g., cell or organism of origin. For the purposes of this disclosure a “polynucleotide sequence” can be any genomic DNA, cDNA, or RNA, and can also include protein-nucleic acid complexes of which the DNA or RNA sequence is the primary determinant of specificity. For ease of reference, individual components of a library are frequently referred to as “members” of the library.

The term “gene” is used to refer to any segment of nucleic acid, e.g., DNA or RNA, associated with a biological function. Thus, genes include coding sequences (e.g., for a protein or peptide) and/or the regulatory sequences required for their expression. Genes also include nonexpressed DNA or RNA segments that, for example, form recognition sequences for other proteins. Non-expressed regulatory sequences include, e.g., “promoters” and “enhancers,” to which regulatory proteins such as transcription factors bind, resulting in transcription of adjacent or nearby sequences.

An “exogenous” gene or “transgene” is a gene foreign (or heterologous) to the cell, or homologous to the cell, but in a position within the host cell nucleic acid in which the element is not ordinarily found. Exogenous genes can be expressed to yield exogenous polypeptides. A “transgenic” organism is one which has a transgene introduced into its genome. Such an organism may be either an animal or a plant.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine -conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

A “parental” cell, or organism, is an untransformed member of the host species giving rise to a transgenic cell, or organism. A “host” is the recipient of a transforming vector.

Introduction

The present invention provides methods for identifying and manipulating one or more (and often multiple) components of a pathway, or even several pathways, that contribute to a cellular or organismal phenotype, including a complex or multigenic phenotype. In bacteria, it is frequently the case that functional units (“operons”), composed of several genes, the products of which all contribute to the same metabolic pathway, are spatially arranged in proximity on a chromosome or on an episome such as a plasmid. Indeed, such proximity is also a pertinent feature in the coordinated induction or repression of the multiple gene products making up the pathway.

However, in many eukaryotes, especially multicellular eukaryotes such as many plant and animal species of commercial and/or agronomic interest, the several genes that contribute to a given metabolic or genetic pathway are often dispersed throughout the genome, only infrequently being arranged in proximity within the genome, and even less frequently being subject to any coordinated regulatory effects due to that proximity.

This disconcerted regulation and disparate localization present formidable obstacles to the controlled regulation of complex phenotypes that are the result of metabolic and genetic pathways, and very often the result of several such pathways acting together. The present invention provides methods for identifying multiple elements of a pathway, and concentrating them locally on one or more episomal vectors, or concentrating regulators of such elements (e.g., antisense sequences, peptide modulators). The multiple elements, or element modifying factors, can then optionally be evolved, synchronously, and selected based on their cumulative effects on a complex phenotype. Because the selected vectors are readily manipulated in vitro, and in bacterial, or eukaryotic cell culture, the rate at which appreciable genetic change can be achieved is significantly enhanced compared to the rates at which eukaryotic genomes typically evolve, e.g., in standard breeding and selection methods. Furthermore, these methods make it possible to exert control over complex phenotypes that require regulation at multiple points in a metabolic or genetic pathway.

The present invention, while providing novel methods that are particularly well suited to the regulation of complex phenotypes or traits, also offers significant advantages in applications aimed at regulating traits controlled by a single metabolic or genetic target. For example, the invention provides methods for rapidly identifying and improving regulators of unknown targets involved in a phenotype of interest. The present invention, by taking advantage of the spatial concentration of potential regulators, also provides a simple and rapid means for screening and optimizing peptides that modulate the activity of cellular targets, such as enzymes, binding proteins and the like.

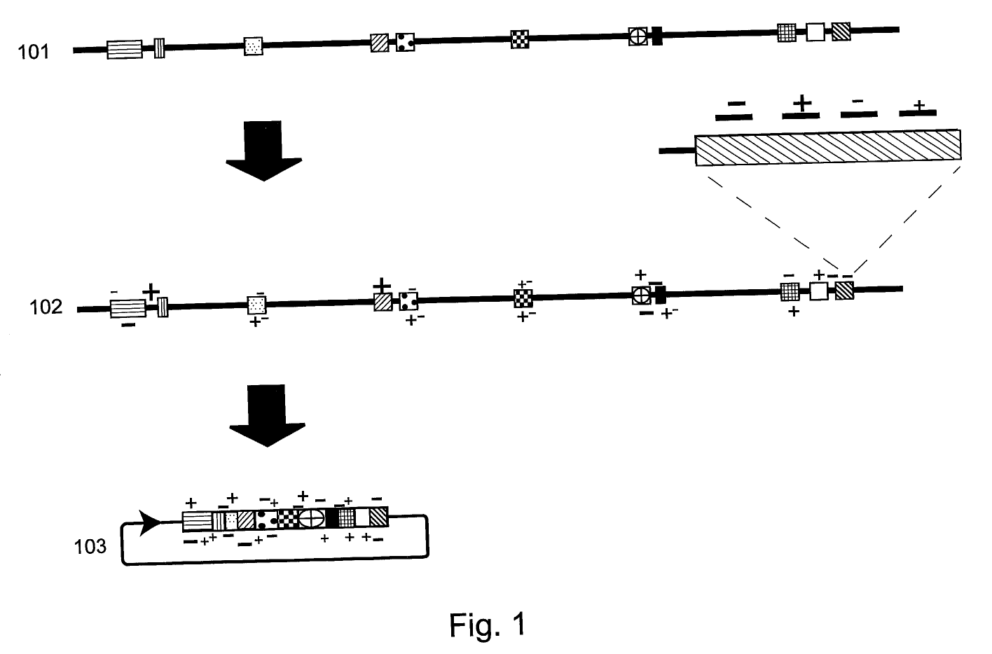

In one illustrative embodiment depicted in FIG. 1, multiple short genetic elements (e.g., typically ranging in size between about 15 and about 1000 bp, e.g., more typically between about 15 and about 200 bp, or, e.g., between about 15 and 150 bp, or between about 20 and about 100 bp or, e.g., between about 20 and about 50 bp) (102) corresponding to several (or a few or many) genes in a genetic or metabolic, e.g., biochemical, pathway (101) that contribute to a complex phenotype, are joined together on an episomal vector (103). In some embodiments, multiple elements (e.g., between about 2 and 10, or about 3-6, or about 4) corresponding to a single gene are included on the same episomal vector. The individual elements can be segments of the genes comprising the genetic or metabolic pathway, or alternatively, they can be regulatory or modifying factors such as antisense suppression elements, sense suppression elements, ribozymes, tRNAs, components of RNPs, or elements encoding structural proteins such as transcription factors, e.g., trans-dominant, dominant-negative, peptide modulator, or decoy molecules.

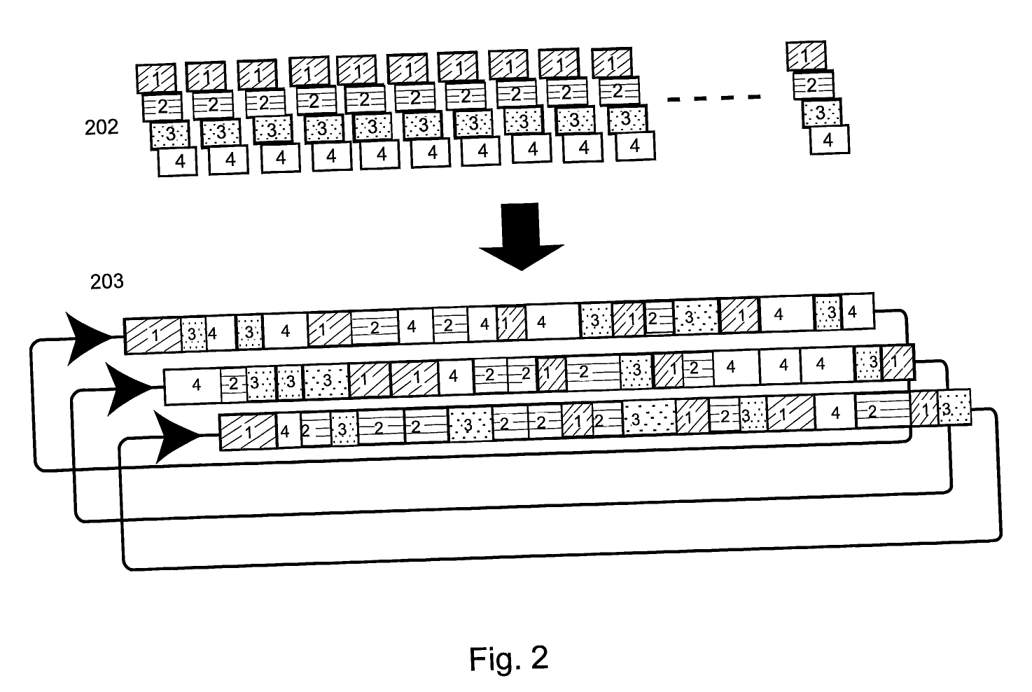

Different elements, and combinations of elements are joined together, e.g., by ligation, on members of a population of episomal vectors to produce a population (e.g., a library) of conjoint polynucleotide segments, as illustrated schematically in FIG. 2. Depending on the size and structural characteristics of the individual elements, expression of the elements is under unified regulatory control, e.g., under the control of a single promoter and/or enhancer. Alternatively, multiple promoters and/or enhancers, e.g., one promoter per element, is utilized to coordinate expression. In general, shorter gene segments are placed under the regulatory control of one or a few promoters, while it is preferable to independently regulate larger genetic elements.

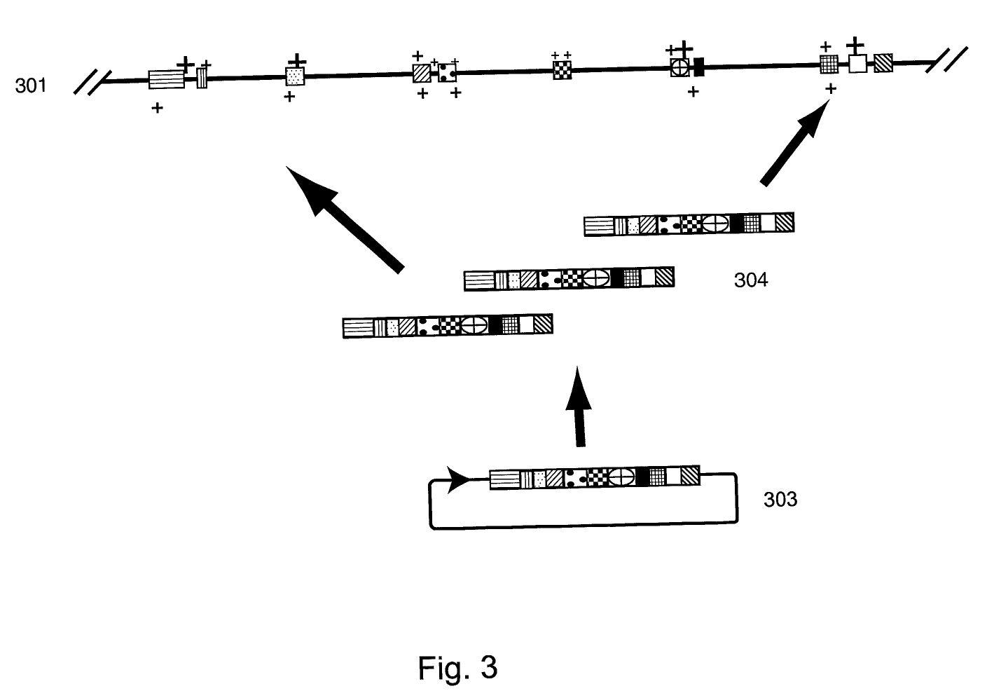

Members of the library of conjoint polynucleotide segments are introduced (e.g., transfected, transformed, transduced, infected, etc.) into an appropriate prokaryotic or eukaryotic host cell for expression and selection. In this manner, episomal vectors that control (e.g., influence, regulate, or modify) complex phenotypes are identified. If so desired, episomal vectors that confer a desired phenotype, (i.e., meet specified selection or screening criteria) are recovered and optionally subjected to one or more diversifying procedure (FIG. 3), e.g., recombination, recursive sequence recombination, mutagenesis, etc. to produce recombinant or variant concatamers. These diversified concatamers are then subjected to additional rounds of screening and/or selection until an optimized set of elements (gene segments, regulatory elements, modifying elements, etc.) are identified.

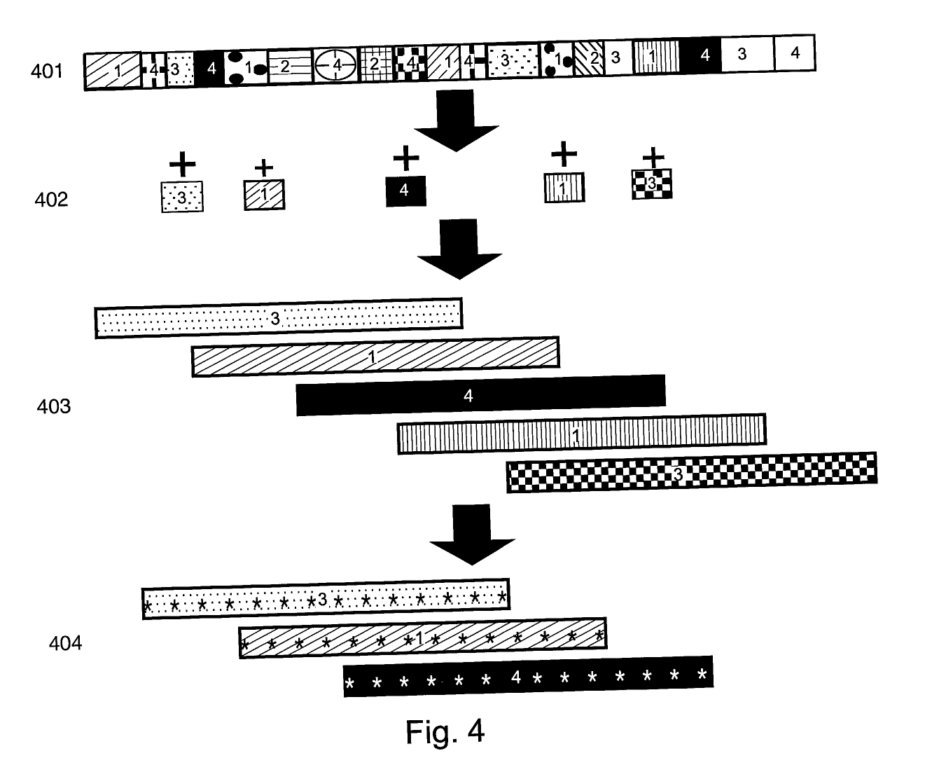

Optionally, as illustrated in FIG. 4, the individual elements that compose the selected (e.g., best) recombinant concatamers (401), are recovered and utilized, e.g., as hybridization probes (402), to isolate the individual genes (403), e.g., cDNAs, minigenes, or genomic DNAs, including the respective regulatory regions, that underlie the desired complex phenotype. Such full length or partial genes, and/or their respective regulatory regions can also be subjected to a variety of diversification procedures to produce optimized variants of the genes of interest.

Multigenic Phenotypes

Classical genetics is largely focused on understanding and manipulating phenotypes that are the result of a single genes (referred to as single gene traits). Such single gene traits exhibit readily appreciable differences in phenotype based on the alleles or combinations of alleles at a single genetic locus. Many human genetic diseases, including cystic fibrosis, and sickle cell anemia, among the more common, are the result of mutations in a single gene (e.g., mutations in a chloride channel, and hemoglobin, in cystic fibrosis and sickle cell anemia, respectively). However, the vast majority of the diseases affecting humans are multigenic in nature. That is, they are a function of numerous spatially separated genetic loci, the products of which interact in multiple genetic and/or metabolic pathways to result in a complex phenotype, which, often depending on environmental circumstances, is perceived as disease.

Similarly, many of the traits of commercial interest, e.g., in plants, fungi, animals or bacteria, are multigenic traits. Indeed, in spite of the fact that traits such as those Mendel originally selected in the common garden pea, each of which was subject to independent and simple genetic control, the vast majority of traits of agronomic interest in agricultural species are multigenic traits that are under the influence of numerous and complex interactions between multiple genes and their products.

Many complex phenotypes can be described in numerical terms. That is, variation between individuals can be assigned a numerical value and the differences within a population can be described in quantitatively. For example, yield, height at maturity, time to germination, growth rate, and time to maturity, are traits of agronomic interest in many crop species, e.g., corn, wheat, sunflower, etc., that are easily described in numerical terms. The genes that control such quantitative traits are often designated “quantitative trait loci” (QTL).

However, many phenotypes of interest, are not described adequately in one dimensional numerical terms. For example, while the overall lipid content present in a grain, can be represented in numerical terms, the often complex mixtures that contribute to the nature of the lipid composition are more complex. Not surprisingly, such complex phenotypes are often the product of multiple, related and even unrelated metabolic and/or genetic pathways. It is, therefore, often difficult to manipulate such complex phenotypes with predictable, easily quantifiable and desirable results.

Further exploring the example of lipid content of a grain, several types of manipulations can be desirable. For example, in addition to increasing or decreasing the overall lipid content, altering the lipid profile, e.g., to produce fatty acids, oils or fats not previously produced by the species, or in different ratios in the species, can be desirable. Because the lipid profile is a function of multiple gene products, including transcription factors that regulate single or multiple lipid synthetic enzymes, enzymes that regulate conversion of carbon sources to fatty acids, enzymes (e.g., fatty acid synthases, transacylases, condensing enzymes, thioesterases, etc.) that catalyze compositional changes in fatty acids, and carrier proteins that act as cofactors in plastid lipid biosynthesis, among many others, it is necessary to make multiple metabolic changes in a concerted fashion to effect an alteration in the lipid profile. Additional details regarding genes and pathways involved in lipid metabolism in plants can be found, e.g., in WO 00/61740 “Modified Lipid Production” by Yuan et al.

In some cases, it is necessary to alter the substrate specificity or activity of one or more elements in a pathway to achieve the desired results. In other cases, key molecular switches can be manipulated. Examples of molecular switches include transcription factors that regulate one or more elements of the pathway. Other examples include enzymes that act at critical regulatory branchpoints, e.g., metabolic “bottlenecks.” For example, in the case of fatty acid synthesis, a key branch point exists between synthesis of membrane lipids and synthesis of storage fats. This switch is controlled by the acyl-CoA:diacylglycerol transferase enzyme (DAGAT). In yet other cases, feedback loops, resulting in the inhibition of a key step in the pathway by a product of that pathway act as molecular switches.

In many cases, the relevant changes are regulatory in nature. For example, by increasing the level of e.g., medium chain thioesterases, while effecting a simultaneous decrease in stearoyl-ACP and/or oleoyl ACP-thioesterases, the composition of the resultant fatty acid can be shifted to shorter carbon backbones. Such an alteration can be accomplished by mutating structural genes, or by altering regulatory aspects of the target genes. For example, mutations in the promoter regions of the genes can alter the expression level of the related structural gene. In addition to regulation at the transcriptional level, gene expression can be regulated at the DNA level: e.g., chromatin structure; methylation; amino-acylation, the RNA level: e.g., induction/repression of transcription; splicing, including alternative splicing, and the protein level: post-translational modification, protein turn-over.

Many of these regulatory mechanisms are epigenetic in nature. That is, they exert their effect not through alterations, i.e., mutations, in the base composition of the gene, but rather through, so called “paramutations,” which while, frequently heritable, are often unstable. Epigenetic mechanisms include: chromatin silencing, methylation (see, e.g., Russo et al. (eds.) Epigenetic Mechanisms of Gene Regulation CSHL Press, Cold Spring Harbor), amino-acylation (Jacobs and Holt (2000) Hum Mol Genet 9:463), and DNA recombination (Roy and Runge (2000) Curr Biol 10: 111), cytoplasmic effects such as maternal effects, antisense and sense suppression, cosuppression, post-transcriptional gene silencing and others.

The present invention takes advantage of several related epigenetic mechanisms, that act at the transcriptional and post-transcriptional level, to produce rapid, broadly adaptable methods for identifying and manipulating complex phenotypes such as yield, protein composition, lipid content, and the like. In particular, mechanisms that result in gene silencing at the transcriptional, post-transcriptional, and post-translational level are employed, including: sense suppression, cosuppression, antisense suppression, and post transcriptional suppression, terms which describe an overlapping and related set of regulatory events.

Post-transcriptional Suppression

Observed primarily in plants, sense suppression and cosuppression refer to the phenomenon observed variously in cases where a transgene possessing a strong promoter or viral vectors carrying sequences with homology to endogenous sequences result in phenotypes that are often the opposite of those expected. That is, they produce an apparent knock-out effect rather than overexpression. It has been proposed (e.g., Jorgensen et al. (1996) in Epigenetic Mechanisms of Gene Regulation, Russo, Martienssen and Riggs, eds., pp393-402; Baulcombe (1999) Current Opinion in Plant Biology 2:109) that this is the result of an RNA-mediated defense (RMD) mechanism that protects plants against viruses.

Expression of transgene or virus-related sequences above a threshold level results in a post-transcriptional cytoplasmic event, which results in a sequence specific turnover process that suppresses gene expression. Also acting at a post-transcriptional level, antisense suppression results in inhibition of expression of sequences complementary to the sequences expressed by the transgene and/or virus. Either sense or antisense (or combinations of the two) suppression mechanisms can be used to probe complex phenotypes, and to manipulate the genes and pathways responsible.

Post-translational Regulation

In addition to such mechanisms as sense suppression, cosuppression and antisense suppression that act at the level of transcription, a variety of regulatory tools which act as, or act at the level of, encoded proteins or peptides are available and adapted to the methods of the present invention. For example, dominant-negative polypeptides (or peptides) when expressed in a cell along with a cellular counterpart or cognate protein, are capable of inhibiting activity of the cognate protein. Such dominant-negative proteins can act in a variety of manners. In some cases, dominant-negative variants include binding domains and are capable of interacting with a cellular cognate inducing an inactive (or preventing an activating) conformational change. In other cases, a dominant-negative competitively binds to a substrate, preventing binding of the substrate to the cellular cognate. More broadly, any transdominant protein or peptide (or perturbagens, see, e.g., Caponigro et al. (1998) Proc. Natl. Acad. Sci. USA 95:7508-13) that modulates function of a protein, whether a cognate or not, can be employed. Alternatively, peptide modulators, such as peptide inhibitors, can bind competitively (e.g., blocking a substrate or ligand binding site) or allosterically (e.g., inducing an inactivating conformational change), thus, modifying the activity level of a cellular target contributing to a phenotype of interest.

Cellular Targets

As described above, the present methods are applicable to a wide variety of phenotypes, whether due to a single, e.g., unknown, gene or protein, or to multiple genes or proteins, in one or more genetic or metabolic pathway, which have previously been controlled with only limited success. In particular, traits of agronomic interest are especially well-suited to the present methods. Such traits include: oil content or composition, fat content or composition, sugar content or composition, starch content or composition, protein content or composition, phytochemical content or composition, nutraceutical content or composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity.

While particularly suitable for the analysis and regulation of complex phenotypes in plants, various adaptations, most particularly those using antisense sequences, are readily adaptable to other organisms, including archae-bacteria, yeast, fungi, and animals. For example, traits such as timing and/or quantity of production of secondary metabolites, resistance to toxicity by secondary metabolites, and viability and/or metabolic activity in organic or other solvents, is of commercial interest in industries employing bacterial, yeast or fungal fermentation processes.

In one preferred embodiment, elements of pathways involved in desulfurization and refinement of petroleum are targets of the present invention. For example, desulfurization of oil during refinement is an appealing target of bioremediation by microorganisms having enhanced abilities, e.g., to catabolize dibenzothiophene, produced by the methods of the present invention. Starting materials include known genes, e.g., the soxA, soxB, and soxC (dszA, dszB, dszC: U08850) genes of Rhodococcus rhodochrous, as well as unselected sequences from various Rhodococcus and other species. Other chemical reactions relevant to the refining and processing of petroleum products which are targets of the invention include but are not limited to alkene epoxidation, alkane oxidation (alkane hydroxylation), aromatic hydroxylation, dealkylation of alkylamines, dealkylation of reduced thio-organics, dealkylation of alkyl ethers, oxidation of aryloxy phenols, oxidation of π-bonds, dehydrogenation, decarbonylation, and oxidative dehalogenation. One preferred example of such a target includes Cytochrome P450, e.g., SubC (CYP105A1, CYP105B1) of Streptomyces griseolus. Additional details regarding genetic and metabolic pathways relevant to the desulfurization and refinement of petroleum products, as well as numerous other pathways of interest, are found, e.g., in U.S. Pat. No. 5,837,458 “METHODS AND COMPOSITIONS FOR CELLULAR AND METABOLIC ENGINEERING” to Minshull et al. WO 00/09682 “DNA Shuffling of Monooxygenase Genes for Production of Industrial Chemicals” by Affholter et al., and WO 01/12791 “DNA Shuffling of Dioxygenase Genes for Production of Industrial Chemical” by Selifonov.

Similarly, traits of interest in the breeding and production of animal species are also amenable to the methods of the invention. Such traits include, but are not restricted, to growth rate, lean body mass indices, metabolic efficiency, disease resistance, and the like. Furthermore, numerous traits (e.g., blood pressure, glucose metabolism, etc.) related to human health and disease can be investigated using in vitro cell culture techniques and animal models, according to the present methods. The results of such studies provide useful insight into potential targets for pharmaceutical intervention.

Typically, the phenotypes of interest, such as those described above, are the products, directly or indirectly, of one or more cellular target. Such cellular targets include, e.g., any of the enzymes, transcription factors, hormones, receptors, etc., involved in the genetic or metabolic pathway or pathways contributing to the phenotype. These targets are the subject of regulation or modulation by the nucleic acids, or products encoded by the nucleic acids, of the present invention, as described in further detail below, and in the Examples.

Identification and Regulation of Phenotypes by Episomes

The present invention utilizes episomal constructs to identify and manipulate complex, multigenic phenotypes to achieve desired phenotypic improvements. Traditionally, improvements in valuable plant and animal species have been the product of selective breeding, e.g., hybridization, programs. Such approaches, while in many cases resulting in significant phenotypic improvements, are generally slow, expensive and laborious. This is largely because they operate at the level of an intact organism, and each cycle of breeding and selection is fixed by the generation time of the organism in question.

In recent years, molecular methods such as transgenic techniques (including, e.g., knock-ins, knock-outs) have been employed, in prokaryotes and in eukaryotes, including both plants and animals, to produce organisms with improved characteristics.

Traditional hybridization approaches offer the benefit that little information regarding the underlying genetic and/or metabolic pathway is required. In addition, multiple elements of the pathway can be selected for simultaneously, as it is the end-product phenotype that is the point of selection. However, because the entire genetic background is the subject of selection, deleterious effects often counterbalance the desirable effects, reducing the overall success and efficiency of the program.

Conversely, transgenic approaches permit the manipulation of a single gene, or small set of genes. This approach offers the benefit of reducing the time required to the span of a single generation. Still, the drawback remains that it is often difficult to predict with certainty, the ultimate phenotypic result of a given transgene.

The present invention provides means to identify elements of a genetic or metabolic pathway in a coordinated fashion. Furthermore, the invention provides methods for evolving the components, or regulators of those components (e.g., antisense regulators, sense suppressor elements, ribozymes, transcription factors, etc.), in a concerted manner, and subsequently transferring them into a host organism to achieve desirable phenotypic alterations. The following aspects of the invention will be discussed sequentially (i) The use of episomal vectors to identify one or more, e.g., multiple, elements of a genetic or metabolic pathway; (ii) evolution of the vectors to achieve desired phenotypic traits; (iii) and introduction of the evolved vectors into host cells, and organisms to produce phenotypic improvements.

Methods for the Identification of Elements of Complex Phenotypes

Episomes are defined as autonomously replicating vectors that are capable of chromosomal integration. Episomes include plasmids, viruses (including proviruses), bacteriophage, phagemids and artificial chromosomes (such as BACs, YACs and PLACs), and for the purposes of this invention, many transposons, and in some cases Agrobacterium T-DNAs. Exemplary vectors are provided in, e.g., PCT/US00/32298 “Shuffling of Agrobacterium Genes, Plasmids and Genomes for Improved Plant Transformation” by Castle et al., and PCT/US00/32289 “Homologous Recombination in Plants” by Lassner et al., incorporated herein by reference. The present invention takes advantage of several beneficial properties of episomal vectors, to identify multiple genetic elements contributing to a complex phenotype, and to manipulate those elements in a synchronized manner to exert control over a phenotype, including a complex phenotype, resulting in desired characteristics.

In one embodiment, multiple short polynucleotide sequences, or segments, are joined together to form conjoint polynucleotide segments. In some embodiments, the segments are short sense or antisense polynucleotide sequences typically ranging in size from approximately 15 to about 500 bases in length, or from about 15 to about 200 bases, or from about 15 to about 150 bases, or from about 20 to about 100 bases in length, although shorter or longer segments, e.g., cDNAs, minigenes, sequences encoding dominant negative variants, sequences encoding peptide modulators, etc. can also be used. The size and number of elements are often chosen to facilitate subsequent manipulations such as cloning into a vector and/or introducing and expressing the conjoint polynucleotide segments in a host cell. For example, approximately 20 elements, e.g., antisense elements, sense elements encoding peptide modulators, etc., of about 50 nucleotides will result in conjoint polynucleotide segments approximately 1 kilobase in length. In many cases, the number and size of elements are chosen to produce conjoint polynucleotide segments of approximately 4 to about 5 kb in length, e.g., to facilitate cloning into commonly available expression vectors.

Typically, to facilitate manipulation, the multiple segments are placed under regulatory control of a single promoter and/or enhancer selected to control expression in the cell type (or organism) of interest. Alternatively, each segment can be placed under independent regulatory control. The short polynucleotide sequences can be DNA or RNA, and expressed in either the sense (coding) or the antisense (“anticoding”) direction. Alternatively, the polynucleotide segments can be e.g., cDNAs, minigenes, genomic DNA segments, or synthetic DNA sequences such as randomly selected aptamers, random or partially random N-mers, or synthesized consensus sequences. In other embodiments, DNA molecules encoding RNA molecules including ribozymes, tRNAs, components of RNPs, and components of the enzymatic splicing machinery can be used. Alternatively, DNA molecules encoding structural proteins, or domains or subsequences thereof, of such cellular targets as transcription factors, e.g., zinc finger proteins, enzymes, receptors, polypeptide hormones, and the like are employed. In some instances, sequences that are not expressed in a mature protein, e.g., introns, inteins, are included among the elements of conjoint polynucleotide segments.

In some embodiments, multiple conjoint polynucleotide segments are introduced into cells in a combinatorial manner. For example, various combinations of individual elements can be introduced into cells to determine which subsets of elements, all belonging to the same “superset” of elements, provide the desired phenotypic alterations. Alternatively, different combinations of supersets, of which each superset includes different (potentially overlapping) combinations of elements can be introduced into cells as conjoint polynucleotide segments to determine which elements control the phenotype of interest in the desired way. Optionally, both approaches can be employed to identify a set of elements that favorably influence a phenotype of interest.

In alternative embodiments, individual genetic elements (i.e., one or more polynucleotide segments) are introduced on separate episomal elements in combinatorial fashion, and screened or assayed to identify sets of (again, often overlapping) elements that contribute to or influence the desired phenotype of interest. For example, a library of nucleic acids that include one or more polynucleotide segments corresponding to various genetic elements, as described above, operably linked to sequences capable of regulating transcription, is introduced (e.g., transformed or transfected) into recipient cells such that subsets of two or more members of the library are introduced into at least a subset of the recipient cells. In this manner, overlapping subsets of library members, some of which are capable of favorably altering expression or activity of one or more components of a complex or multigenic phenotype, are evaluated as “pools,” and those subsets able to exert the desired effect on the phenotype of interest can be selected, recovered, and/or further manipulated (e.g., recombined, mutated, etc.) at the discretion of the practitioner.

In some embodiments, multiple genetic elements that exist in nature as linked segments of a polynucleotide are utilized to investigate and/or influence a complex phenotype. One example of such an embodiment is the use of viruses, such as polio, or other picornaviruses, which repress cap-dependent translation while enhancing cap-independent translation of mRNA, thus, simultaneously altering multiple metabolic and/or genetic pathways. Similarly, retroviruses carrying oncogenes are able to reverse transcribe, insert themselves into a host genome and express the oncogene which alters multiple genetic and metabolic pathways to effect the complex phenotypic changes associated with transformation and immortalization. Such viruses are adapted to modify the biochemistry, physiology and genetics of their hosts, influencing a variety of pathways that contribute to complex cellular and organismal phenotypes. Accordingly, many viruses provide favorable substrates for the methods of the present invention. Such viruses can be used intact as substrates, e.g., by recombining or mutating selected viral genomes. Alternatively, individual components, or polynucleotide segments corresponding to subsequences therefrom can serve as the substrates for the methods described herein.

In many cases, it is desirable to utilize a vector comprising DNA for certain of the manipulations, and to rely on a transcribed RNA for other aspects of the process. For example, many plant viruses consist of an infectious RNA molecule. When utilizing such a vector, initial cloning and ligation steps, as well as mutagenesis and recombination, steps are frequently performed using a complementary DNA (cDNA) molecule. Transcribed RNA is then used to transduce the appropriate cell or organism.

The DNAs selected can be random (genomic, cDNA or synthetic DNA, e.g., synthetic oligonucleotides comprising random or partially randomized N-mers). That is, the function need not be known in advance. RNA can be isolated from a cell, tissue or organism that is known or suspected to express the relevant factors of interest, or to exhibit a phenotype of interest. For example, to identify key elements regulating lipid composition, RNA derived from oil producing cells can be reverse transcribed using random primers to generate cDNA molecules. These cellular cDNAs are then ligated, under conditions that favor multiple insertions/vector, into an episomal vector under the regulatory control of a strong promoter.

Pre-selection of Polynucleotide Segments

In addition to the random polynucleotide segments described above, numerous methods can be used to pre-select a desired subset of polynucleotide segments or encoded peptides or polypeptides from a library or pool of DNA, RNA or amino acid sequences. It will be apparent to one of skill in the art that the initial library or pool of DNA sequences can itself be either random (e.g., random or partially randomized N-mers, etc.) or selected by any sequence, structural or functional methods available, e.g., as exemplified below.

For example, various methods and genetic algorithms (GAs) known in the art can be used to detect homology or similarity between different polynucleotide sequences. Thus, different types of homology and similarity can be detected and recognized. With an understanding of double-helix pair-wise complement interactions among 4 principal nucleobases in natural polynucleotides, models that simulate annealing of complementary homologous polynucleotide sequences can also be used as a foundation of sequence alignment or other operations typically performed on character strings corresponding to the sequences herein (e.g., word-processing manipulations, construction of figures comprising sequence or subsequence character strings, output tables, etc.). An example of a software package with GAs for calculating sequence similarity is BLAST, which can be adapted to the present invention by inputting character strings corresponding to polynucleotide sequences corresponding to, e.g., genes, cDNAs, components of conjoint polynucleotide segments, and the like.

Alternatively, computational methods such as the WIT (What is there?) system developed by Overbeek et al. (2000) Nucleic Acids Res. 28: 123, that utilize gene sequence and genomic location data to infer structure and function, can be employed. Similarly, methods that utilize sequence sampling and alignment programs, such as AlignACE, Hughes et al. (2000) J. Mol Biol. 296:1205, can be used to identify gene segments of potential relevance.

Similarly, methods that perform computational expression analysis can be used to identify motifs relevant to common regulatory sequences. (see, e.g., Roth et al. (1998) Nat Biotechnol 16:939; Roberts et al., (2000) Science 287:873). Mathematical modeling of metabolic pathways and flux analysis techniques can also be employed to pre-select the sequences of the present invention. For example, publicly available programs such as DBsolve 5.00 (http://websites.ntl.com) and described in Goryanin et al. (1999) Bioinformatics 15:749, can be used to pre-select sequences of interest, or to identify regulatory proteins that interact with these sequences. A wide variety of methods for selecting sequences based on structural, functional and/or statistical information are found in, e.g., WO 00/42560. These methods can be applied to the present invention to pre-select libraries or library components.

High throughput methods for expression analysis, e.g., utilizing cDNA or oligonucleotide arrays, are also favorably used to pre-select candidate sequences. For example double stranded oligonucleotides or cDNA fragments fixed to a matrix can be used to identify interacting protein binding domains (see, e.g., Bulyk et al. (1999) Nat Biotechnol 17:573).

A variety of in vitro and in vivo display methods are also known, and can be adapted to the present invention. Such methods are particularly well adapted to embodiments involving expressed peptides, polypeptides or proteins, e.g., peptide modulators, dominant-negative and transdominant proteins or variants. For example, ribosomal display methods (see, e.g., Jermutus et al. (1998) Current Opinion in Biotechnology, 9:534-548, and references cited therein, can be used to display peptides and proteins in vitro in a cell-free system, e.g., using extracts isolated from, for example, E. coli. Alternatively, numerous well-known systems are available for expressing peptides, protein domains, and the like, for display, e.g., on the surface of Phage (typically as a fusion to a coat protein), bacteria and yeast (e.g., as a fusion with a cell surface protein, such as bacterial OmpA. Displayed peptides or proteins can be detected, for example, by flow cytometry (for useful procedures and protocols, see, e.g., Owens and Loken (1995) Flow Cytometry Principles for Clinical Laboratory Practice, Wiley-Liss, New York; Flow Cytometry: A Practical Approach, 2^{nd} ed (1994) Ormerod (Ed.), IRL Press, Oxford; and Flow Cytometry Protocols: Methods in Molecular Biology, Vol. 91 Jarosqeski and Heller (Eds.) (1997) Humana Press.

In addition, sequences of interest can be selected based on well established methods such as traditional mutagenesis analysis, yeast two hybrid analysis (see, e.g., Chien et al (1991) Proc Natl Acad Sci USA 88:9578; Fields and Song (1989) Nature 340:245) and reverse genetics methods such as gene knockouts.

In summary, many techniques are available to pre-select polynucleotide sequences useful in the present invention, as starting material for identifying elements of a complex metabolic or genetic pathway. Regardless of whether random or pre-selected sequences are utilized the invention can be utilized to identify multiple components and to improve upon phenotypes by controlling the relevant pathways.

Episomal Suppression of Genetic Elements

Libraries of conjoint polynucleotide segments comprising populations of random and/or pre-selected polynucleotide segments joined together as described above are produced and introduced into bacterial or eukaryotic cells of interest. In certain embodiments, the cells are plant cells. Members of the libraries, each consisting of a multiple polynucleotide segments joined together under the operative control of one or several coordinated regulatory sequences, are transduced (transformed, transfected, infected, etc.) into the appropriate recipient cell. Upon expression of the introduced sequences, multiple endogenous genes are suppressed by any of the above described mechanisms, including sense suppression, antisense suppression, transcript cleavage, trans-dominant expression, expression of peptide modulators, use of molecular decoys, etc., as described herein.

By joining multiple antisense or sense segments in a single episome, or in a few co-transfected episomes, multiple genes, acting in one or multiple pathways can be investigated simultaneously. After introduction of the conjoint polynucleotide segments, and expression of the component antisense or sense strand RNAs, multiple endogenous gene sequences are suppressed by either antisense or sense suppression mechanisms. The resulting effect is then analyzed at the phenotypic level, and independent cells exhibiting desirable phenotypic alterations are selected. This permits, analysis and selection of phenotypes that can not be appreciably altered using a single gene approach. Thus, the present invention provides a means of rapidly exploring all accessible phenotypes making it possible, in effect, to determine the limits of genetic manipulation. Alternative methods of evaluation, such as those assaying activity or expression of one or more targets contributing to or determining the phenotype, e.g., enzymes, transcription factors, receptors, etc., can be readily performed at the discretion of the practitioner.

In one exemplary class of embodiments, the segments are derived from “antisense libraries.” That is, random or selected cDNAs cloned in the inverted orientation with respect to a promoter, thus producing an “antisense” strand RNA, are cloned directionally into the episomal vector.

Antisense RNA molecules have long been known to inhibit expression of selected genes. A number of references describe anti-sense and sense suppression, including Antisense Strategies, Annals of the New York Academy of Sciences, Volume 600, Eds. Baserga and Denhardt (NYAS 1992); Milligan et al., 9 July 1993, J. Med. Chem. 36(14):1923-1937; Antisense Research and Applications (1993, CRC Press), and Antisense Therapeutics, ed. Sudhir Agrawal (Humana Press, Totowa, New Jersey, 1996) and U.S. Pat. No. 4,801,340.

“Sense suppression” of genes has also been observed. For examples of the use of sense suppression to modulate expression of endogenous genes see, Napoli, et al., The Plant Cell 2:279 (1990) and U.S. Pat. No. 5,034,323.

For post transcriptional suppression to occur, the introduced sequence need not be full length relative to either the primary transcription product or fully processed MRNA. Generally, higher homology can be used to compensate for the use of a shorter sequence. Furthermore, the introduced sequence need not have the same intron or exon pattern, and homology of non-coding segments is equally effective. Normally, a sequence of between about 30 or 40 nucleotides and about 2000 nucleotides should be used, though a sequence of at least about 50 nucleotides is often used, and sequence of at least about 100 nucleotides or more can also be used.

In another example, ribozymes which are catalytic RNA molecules having antisense and endoribonuclease activity that cleave other RNA molecules based on sequence specificity are used. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. General methods for the construction of ribozymes, including hairpin ribozymes, hammerhead ribozymes, RNAse P ribozymes (i.e., ribozymes derived from the naturally occurring RNAse P ribozyme from prokaryotes or eukaryotes) are known in the art. Castanotto et al. (1994) Advances in Pharmacology 25:289 provides an overview of ribozymes in general, including group I ribozymes, hammerhead ribozymes, hairpin ribozymes, RNAse P, and axhead ribozymes.

The portion of a nucleic acid encoding the ribozyme which is complementary to a target RNA 3′ of the cleavage site on the target RNA, i.e., the ribozyme nucleic acid sequences 5′ of the ribozyme nucleic acid subsequence which aligns with the target cleavage site is often referred to as a “helix 1” ribozyme domain. Once a target RNA is identified (e.g., by virtue of a GUC or GUA), and a ribozyme is constructed which cleaves the target in vivo, one of skill can generate many similar targets and ribozymes by performing routine modification of the given targets and ribozymes. Furthermore, as described, e.g., by Hu et al., PCT publication WO 94103596, antisense and ribozyme functions can be combined in a single oligonucleotide.

In an alternative embodiment, DNA or RNA molecules that are decoy nucleic acids, i.e., nucleic acids having a sequence recognized by a regulatory nucleic acid binding protein (e.g., a transcription factor, cell trafficking factor, etc.). Upon expression, the transcription factor binds to the decoy nucleic acid, rather than to its natural target in the genome. Useful decoy nucleic acid sequences include any sequence to which, e.g., a cellular transcription factor binds.

In another embodiment, nucleic acids that encode proteins that act as dominant negative forms of a protein, and nucleic acids that encode a protein whose phenotype, when supplied by transcomplementation, will overcome the effect of the native form of the protein, so called “transdominant” nucleic acids, are favorably encoded by the conjoint polynucleotide segments of the invention. In still other embodiments, peptides, typically corresponding to short sequences of amino acids rather than to entire domains or proteins, are employed. Such peptide modulators, e.g., peptide inhibitors, can vary in size, but typically do not represent substantially the entire protein from which they are derived or to which they correspond. For example, such peptide modulators are typically from about 5 to about 50 amino acids in length, (e.g., from about 5 to about 100, or even up to about 150, or about 200 amino acids, or more) in length. Peptide modulators bind to a cellular target, such as an enzyme, for example, within the substrate binding site (i.e., peptide inhibitors) or at an alternative site that effects an allosteric change in target conformation that inhibits or enhances activity of the target (i.e., peptide inhibitors and peptide enhancers, respectively).

Evolution of Epigenetic Episomes

In the present invention, nucleic acid constructs can optionally be modified before or after selection for one or more effects. That is, after initial construction of one or more chimeric nucleic acid comprising conjoint polynucleotide segments which encodes one or more factor (anti-sense molecule, ribozyme, sense suppressive molecule, trans-dominant nucleic acid, peptide modulator, molecular decoy, etc.) which can regulate or otherwise influence a metabolic or genetic pathway of interest, as described herein, the chimeric nucleic acid can be diversified to provide a library of related recombinant or variant concatamers, e.g., by one or more diversity generating procedures, prior to screening the chimeras for any desired property. Alternatively, the conjoint polynucleotide segments can be screened in an appropriate system (e.g., a cell or organism such as a fungus or plant), and the nucleic acids then diversified, e.g., by one or more diversity generating procedure to generate a library of recombinant concatamers which is then screened for a trait or property of interest. As an alternative to, or in combination with the diversification of elements arranged as conjoint polynucleotide segments, individual elements, e.g., identified as components of conjoint polynucleotide segments, or through combinatorial analysis of individual elements, can be diversified and screened by a variety of procedures for increasing diversity and identifying favorable variants of a nucleic acid or polypeptide.

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, i.e., recombinant or variant concatamers, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property, e.g. influence on a complex phenotype. This can include identifying any activity that can be detected, for example, in an automated or automatable format, by any of the assays in the art as described below. A variety of related (or even unrelated) properties can be evaluated, in serial or in parallel, at the discretion of the practitioner.

Descriptions of a variety of diversity generating procedures for generating modified nucleic acid sequences, including the recombinant concatamers of the invention, are found the following publications and the references cited therein: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Similarly, other methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection;” WO 00/00632, “Methods for Generating Highly Diverse Libraries;” WO 00/09679, “Methods for Obtaining in Vitro Recombined Polynucleotide Sequence Banks and Resulting Sequences;” WO 98/42832 by Arnold et al., “Recombination of Polynucleotide Sequences Using Random or Defined Primers;” WO 99/29902 by Arnold et al., “Method for Creating Polynucleotide and Polypeptide Sequences;” WO 98/41653 by Vind, “An in Vitro Method for Construction of a DNA Library;” WO 98/41622 by Borchert et al., “Method for Constructing a Library Using DNA Shuffling;” WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” PCT/US00/26708 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

In brief, several different general classes of sequence modification methods, such as mutation, recombination, etc. are applicable to the present invention and set forth, e.g., in the references above. That is, the conjoint polynucleotide segments of the invention can be diversified by any one or more of the above referenced techniques, as further described below, to create a diverse set of recombinant concatamers, which can be screened or selected for a desired phenotype.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process, and many process variants, are described in several of the references above, e.g., in Stemmer (1994) Proc. Natl. Acad. Sci. USA 91:10747-10751. Thus, the conjoint polynucleotide segments can be fragmented and recombined in vitro to produce libraries of recombinant concatamers.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Thus, conjoint polynucleotide segments can be transformed into cells, e.g., using viral vectors, and allowed to undergo recombination in vivo.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., WO 00/04190 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Olgonucleotide Mediated Nucleic Acid Recombination;” PCT/US00/26708 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for recombination of the sequence elements corresponding to conjoint polynucleotide segments in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775, filed Sept. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity into the conjoint polynucleotide segments of the invention. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 “Methods for Generating and Screenig Novel Metabolic Pathways”) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to pre-select or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 5 IF to lambda repressor” J. Biol. Chem. 264:13355-60); and “Walk-Through Mutagenesis” (Crea, R; U.S. Pat. Nos. 5,830,650 and 5,798,208, and EP Patent 0527809 B1. It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above discussion provides many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

In one aspect, the present invention provides for the recursive use of any of the diversity generation methods noted above, in any combination, to evolve chimeric nucleic acids or libraries of recombinant concatamers that influence one or more multigenic pathway. In particular, as noted, the relevant chimeric nucleic acids which influence, or which putatively may influence one or more multigenic pathway can be modified before selection, or can be selected and then recombined, or both. This process can be reiteratively repeated until a new or improved trait having a desired property is obtained.

Post-diversification Screening Techniques

The precise screening method that is used in the various procedures herein is not a critical aspect of the invention. In general, one of skill can practice appropriate screening (i.e., selection) methods, by reference to the activity to be selected for (e.g., yield, oil content, enzyme activity, etc., e.g., as set forth herein).

In any case, following introduction of chimeric nucleic acids which influence multigenic pathways, and/or following one or more recombination cycle(s) with the chimeric nucleic acids, at least one cycle of screening or selection for chimeras having a desired property or characteristic can be performed. If a recombination cycle is performed in vitro, the products of recombination, e.g., recombinant concatamers, are generally, though not always, introduced into cells before the screening step. Recombinant concatamers can also be linked to an appropriate vector or other regulatory sequences before screening. Alternatively, products of recombination generated in vitro are sometimes packaged in viruses (e.g., bacteriophage or plant viral vectors) before screening. If recombination is performed in vivo, recombination products can sometimes be screened in the cells in which the recombinant concatamer is desirably active (e.g., in plants, fungi, bacteria, yeast, animals, or the like). In other applications, recombinant concatamers are extracted from the cells, and optionally re-packaged before screening.

The nature of screening or selection depends on what property or characteristic is to be acquired or the property or characteristic for which improvement is sought. It is not usually necessary to understand the molecular basis by which particular products of recombination (recombinant concatamers or individual segments thereof) have acquired new or improved properties or characteristics relative to the starting substrates. For example, a multi-genic pathway can have many component sequences, each having a different intended role (e.g., coding sequences, regulatory sequences, targeting sequences, stability-conferring sequences, subunit sequences, sequences affecting chromosome structure, and the like). Each of these component sequences can be tested for independently or simultaneously using available detection methods.

Depending on the particular screening protocol used for a desired property, initial round(s) of screening can sometimes be performed using bacterial cells, which are desirable screening systems due to high transfection efficiencies and ease of culture. However, bacterial expression is often not practical or desired, and plant, yeast, fungal or other eukaryotic systems are also used for library expression and screening. Similarly, other types of screening which are not amenable to screening in bacterial or simple eukaryotic library cells, are performed in cells selected for use in an environment close to that of their intended use. Final rounds of screening can be performed in the precise cell type of intended use.

If further improvement in a property is desired, at least one and usually a collection of recombinant concatamers (or individual elements contributing to, or derived from, a recombinant concatamer) surviving a first round of screening/selection are subject to a further round of recombination. These recombinant concatamers (or individual elements) can be recombined with each other or with exogenous segments representing the original substrates or further variants thereof. Again, recombination can proceed in vitro or in vivo. If the previous screening step identifies desired recombinant segments as components of cells, the components can be subjected to further recombination in vivo, or can be subjected to further recombination in vitro, or can be isolated before performing a round of in vitro recombination. Conversely, if the previous screening step identifies desired recombinant segments in naked form or as components of viruses, these segments can be introduced into cells to perform a round of in vivo recombination. The second round of recombination, irrespective how performed, generates further recombinant segments which encompass additional diversity than is present in recombinant concatamers resulting from previous rounds.

The second round of recombination can be followed by a further round of screening/selection according to the principles discussed above for the first round. The stringency of screening/selection can be increased between rounds. Also, the nature of the screen and the property being screened for can vary between rounds if improvement in more than one property is desired or if acquiring more than one new property is desired. Additional rounds of recombination and screening can then be performed until recombinant concatamers have sufficiently evolved to acquire the desired new or improved property or function.

In an alternative embodiment, the individual segments are maintained on independent episomal units. Multiple episomes are then transformed, in combinatorial fashion, into the appropriate cells, and screened as described above.

In some cases, multiple phenotypes, or multiple aspects describing a phenotype may be screened for simultaneously. In such cases, the results of various screening or other assays can be compiled to generate n-dimensional profiles that account for multiple aspects of a complex phenotype. For example, returning to the example of lipid profile of a grain, parameters include such variables as grain kernel weight, cell density, water content, solids content, total oil content, various parameters describing oil composition, and the like. Standard n-dimensional analysis such as principal component analysis (PCA) can be used to examine and refine the multivariate matrix profile. As the number of variables increases it becomes desirable to perform the analyses with computer assistance. Software for performing multivariate analysis is available from a number of sources and can be adapted to the present invention, e.g., from Partek, Inc. Thus the multivariate matrix profiles of the present invention can be computer generated or other data sets, topological maps or other representations of the products of multivariate analysis. Accordingly, in many cases the results of screening assays, including multivariate matrix profiles are stored in a computer readable medium accessed through data input and output devices, and manipulated, e.g., analyzed, by a processing unit, e.g., CPU, of a computer, e.g., PC, mainframe, etc. Additional details regarding computer access, storage and manipulation of multivariate analysis are provided in, e.g., WO 00/42560 by Selifonov et al. “Methods for Making Character Strings, Polynucleotides and Polypeptides Having Desired Characteristics.”

General texts which describe molecular biological techniques useful herein, including the use of vectors, promoters and many other relevant topics related to, e.g., the cloning and expression of episomes, production of nucleic acids, polynucleotide segments, etc., include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Similarly, examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the nucleic acids of the invention are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

In construction of recombinant episomal vectors of the invention, which include, for example, plasmids or viruses comprising exogenous DNA sequences such as sense sequences, antisense sequences, ribozymes, etc., a promoter fragment is optionally employed which directs expression of a nucleic acid in any cell, intracellular organelle, or in any or all tissues of a regenerated plant, animal or other organism. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various bacterial, plant or animal genes known to those of skill. Alternatively, the promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers.

Any of a number of promoters which direct transcription in cells can be suitable. The promoter can be either constitutive or inducible. For example, in addition to the promoters noted above, promoters of bacterial origin which operate in plants include the octopine synthase promoter, the nopaline synthase promoter and other promoters derived from native Ti plasmids. See, Herrara-Estrella et al. (1983), Nature, 303:209-213. Viral promoters include the 35S and 19S RNA promoters of cauliflower mosaic virus. See, Odell et al. (1985) Nature, 313:810-812. Other plant promoters include the ribulose-1,3-bisphosphate carboxylase small subunit promoter and the phaseolin promoter. The promoter sequence from the E8 gene and other genes may also be used. The isolation and sequence of the E8 promoter is described in detail in Deikman and Fischer, (1988) EMBO J. 7:3315-3327. Many other promoters are in current use and can be coupled to an exogenous DNA sequence to direct expression of the nucleic acid.

If expression of a polypeptide, including various viral, bacterial and exogenous gene products, such as viral coat proteins, biosynthetic enzymes (e.g., including dominant negative, and transdominant variants) and markers of the present invention, is desired, a polyadenylation region at the 3′-end of the coding region is typically included. The polyadenylation region can be derived from the natural gene, from a variety of other plant genes, or from, e.g., T-DNA.

The vector comprising the sequences (e.g., promoters or coding regions) from genes encoding expression products and transgenes of the invention optionally include a nucleic acid subsequence, a marker gene which confers a selectable, or alternatively, a screenable, phenotype on plant cells. For example, the marker may encode biocide tolerance, particularly antibiotic tolerance, such as tolerance to kanamycin, G418, bleomycin, hygromycin, or in plants: herbicide tolerance, such as tolerance to chlorosluforon, or phosphinothricin (the active ingredient in the herbicides bialaphos or Basta). See, e.g., Padgette et al. (1996) “New weed control opportunities: Development of soybeans with a Round UP Ready™ gene” In: Herbicide-Resistant Crops (Duke, ed.), pp. 53-84, CRC Lewis Publishers, Boca Raton (“Padgette, 1996”). For example, crop selectivity to specific herbicides can be conferred by engineering genes into crops which encode appropriate herbicide metabolizing enzymes from other organisms, such as microbes. See, Vasil (1996) “Phosphinothricin-resistant crops” In: Herbicide-Resistant Crops (Duke, ed.), pp 85-91, CRC Lewis Publishers, Boca Raton) (“Vasil”, 1996).

Production of Transgenic Cells and Organisms

The present invention also relates to host cells and organisms which are transformed with vectors, e.g., including recombinant concatamers or individual elements derived therefrom, of the invention, and the production of polypeptides of the invention, e.g., dominant negative or transdominant protein variants, by recombinant techniques. Host cells are genetically engineered (i.e., transformed, transduced or transfected) with the vectors of this invention, which may be, for example, a cloning vector or an expression vector. The vector may be, for example, in the form of a plasmid, an agrobacterium, a virus, a naked polynucleotide, or a conjugated polynucleotide. In some preferred embodiments, the vectors are episomal vectors capable of both autonomous replication and chromosomal integration. The vectors are introduced into bacteria, yeast, fungi, or animal or plant tissues, cultured cells, or in the case of plants, protoplasts, e.g., by standard methods. In addition, the methods of the present invention can be adapted to transformation of a community of organisms such as microbial consortia, sponges, slime molds, and the like. Useful methods well known in the art include electroporation (From et al., Proc. Natl. Acad. Sci. USA 82, 5824 (1985), microinjection, infection by viral vectors such as cauliflower mosaic virus (CaMV) (Hohn et al., Molecular Biology of Plant Tumors, (Academic Press, New York, 1982) pp. 549-560; Howell, U.S. Pat. No. 4,407,956), high velocity ballistic penetration by small particles with the nucleic acid either within the matrix of small beads or particles, or on the surface (Klein et al., Nature 327, 70-73 (1987)), use of pollen as vector (WO 85/01856), or use of Agrobacterium tumefaciens or A. rhizogenes carrying a T-DNA plasmid in which DNA fragments are cloned. The TDNA plasmid is transmitted to plant cells upon infection by Agrobacterium tumefaciens, and a portion is stably integrated into the plant genome (Horsch et al., Science 233, 496498 (1984); Fraley et al., Proc. Natl. Acad. Sci. USA 80, 4803 (1983)). Techniques well known in the production of transgenic cells and animals, can be found in e.g. Hogan et.al., Manipulating the Mouse Embryo, second edition, (1994) Cold Spring Harbor Press, Plainview).

Alternatively, the polynucleotides of the invention can be used to transform intracellular organelles such as mitochondria and chloroplasts. In some cases, complex phenotypes of interest involve genes encoded by mitochondrial and/or chloroplast DNA molecules. Such DNA molecules are suitable for integration by the episomal vectors herein described.

The engineered host cells can be cultured in conventional nutrient media modified as appropriate for such activities as, for example, activating promoters or selecting transformants. In some cases the cells can optionally be used to generate transgenic organisms. The present invention also relates to the production of transgenic organisms, which may be bacteria, yeast, fungi, or plants. A thorough discussion of techniques relevant to bacteria, unicellular eukaryotes and cell culture may be found in references enumerated above and are briefly outlined as follows. Several well-known methods of introducing target nucleic acids into bacterial cells are available, any of which may be used in the present invention. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect plant cells or incorporated into Agrobacterium tumefaciens related vectors to infect plants. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA, Second Edition, Scientific American Books, N.Y.

Transforming Nucleic Acids into Plants.

One class of embodiments pertain to the production of transgenic plants using evolved episomal vectors of the invention. Techniques for transforming plant cells with nucleic acids are generally available and can be adapted to the invention by the use of evolved plasmids, viruses, and components thereof, and by the use of agrobacterium strains comprising evolved vectors. In addition to Berger, Ausubel and Sambrook, useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, U.K. Plant regeneration from cultured protoplasts is described in Evans et al. (1983) Handbook of Plant Cell Cultures pp 124-176, (MacMillan Publishing Co., New York); Davey (1983) Protoplasts pp 12-29 (Birkhause, Basel); Dale, ibid, pp 31-41; and Binding (1985) Plant Protoplasts pp 21-73 (CRC Press, Boca Raton).

The nucleic acid constructs of the invention, e.g., plasmids, viruses, DNA and RNA polynucleotides, are introduced into plant cells, either in culture or in the organs of a plant by a variety of conventional techniques. For example, recombinant DNA or RNA vectors suitable for transformation of plant cells are isolated and/or prepared. To introduce an exogenous DNA, which can be a recombinant or chimeric DNA, e.g., a recombinant concatamer, the exogenous DNA sequence can be incorporated into an episomal vector of the invention and transformed into the plant as indicated above. Where the sequence is expressed, the sequence is optionally combined with transcriptional and/or translational initiation regulatory sequences which direct the transcription (or translation) of the sequence from the exogenous DNA in the intended tissues of the transformed plant.

The DNA constructs of the invention, for example plasmids, can be introduced directly into the genomic DNA of the plant cell using techniques such as electroporation and microinjection of plant cell protoplasts, or the DNA constructs can be introduced directly to plant cells using ballistic methods, such as DNA particle bombardment.

Microinjection techniques for injecting e.g., cells, embryos, and protoplasts, are known in the art and well described in the scientific and patent literature. For example, a number of methods are described in Jones (ed) (1995) Plant Gene Transfer and Expression Protocols—Methods in Molecular Biology, Volume 49 Humana Press Towata N.J., as well as in the other references noted herein and available in the literature.

For example, the introduction of DNA constructs using polyethylene glycol precipitation is described in Paszkowski, et al., EMBO J. 3:2717 (1984). Electroporation techniques are described in Fromm, et al., Proc. Nat'l. Acad. Sci. USA 82:5824 (1985). Ballistic transformation techniques are described in Klein, et al., Nature 327:70-73 (1987). Additional details are found in Jones (1995) supra.

In some embodiments, agrobacterium mediated transformation is used to introduce nucleic acids of the invention into plant cells. Agrobacterium mediated transformation relies on the ability of A. tumefaciens or A. rhizogenes to transfer DNA molecules called T-DNA to a host plant cell. A. tumefaciens and A. rhizogenes are the causative agents of the plant neoplastic diseases crown gall and hairy root disease, respectively. Agrobacteria, which reside normally in the soil, detect soluble molecules secreted by wounded plant tissues through a specialized signal detection/transformation system. In the presence of these chemical signals, agrobacteria attach to the cell walls of wound exposed plant tissues. The agrobacteria then excise and transfer a portion of specialized DNA, designated T-DNA and delimited by T-DNA borders, to the host plant cell nucleus where it is integrated into the chromosomal DNA.

This DNA transfer system can be manipulated to transfer exogenous DNA situated between T-DNA borders to a host plant cell of choice. Agrobacterium-mediated transformation techniques, including disarming and use of binary vectors, are also well described in the scientific literature. See, for example Horsch, et al., “A simple and general method for transferring genes into plants.” Science 233:496-498 (1984), and Fraley, et al., “Expression of bacterial genes in plant cells.” Proc. Nat'l. Acad. Sci. USA 80:4803 (1984) and recently reviewed in Hansen and Chilton, “Lessons in gene transfer to plants by a gifted microbe.” Current Topics in Microbiology 240:22-51 (1998) and Das, “DNA transfer from Agrobacterium to plant cells in crown gall tumor disease.” Subcellular Biochemistry 29: Plant Microbe Interactions:343-363 (1998). These techniques are adapted in the present invention to transform plant cells with nucleic acid, especially the recombinant concatamers and other conjoint polynucleotide segments, of the invention.

Embodiments of the present invention also comprise vectors which are viruses. Viruses are typically useful as vectors for expressing exogenous DNA sequences in a transient manner in host cells, including plant and animal cells. In contrast to methods which results in the stable integration of DNA sequences in the plant genome, viral vectors are generally replicated and expressed without the need for chromosomal integration. In particular, plant virus vectors offer a number of advantages, specifically: DNA copies of viral genomes can be readily manipulated in E. coli, and transcribed in vitro, where necessary, to produce infectious RNA copies; naked DNA, RNA, or virus particles can be easily introduced into mechanically wounded leaves of intact plants; high copy numbers of viral genomes per cell results in high expression levels of introduced genes; common laboratory plant species as well as monocot and dicot crop species are readily infected by various virus strains; infection of whole plants permits repeated tissue sampling of single library clones; recovery and purification of recombinant virus particles is simple and rapid; and because replication occurs without chromosomal insertion, expression is not subject to position effects. These many advantages are exploited by the present invention for the production of improved phenotypes using the nucleic acids of the invention.

Over six-hundred-fifty plant viruses have been identified, and are amenable either directly or indirectly as substrates for the directed evolution processes of the invention. Plant viruses cause a range of diseases, most commonly mottled damage to leaves, so-called mosaics. Other symptoms include necrosis, deformation, outgrowths, and generalized yellowing or reddening of leaves. Plant viruses are known which infect every major food-crop, as well as most species of horticultural interest. The host range varies between viruses, with some viruses infecting a broad host range (e.g., alfalfa mosaic virus infects more than 400 species in 50 plant families) while others have a narrow host range, sometimes limited to a single species (e.g. barley yellow mosaic virus). Host range is among the many traits for which it is possible to select appropriate vectors according to the methods provided by the present invention.

Approximately 75% of the known plant viruses have genomes which are single-stranded (ss) messenger sense (+) RNA polynucleotides. Major taxonomic classifications of ss-RNA(+) plant viruses include the bromovirus, capillovirus, carlavirus, carmovirus, closterovirus, comovirus, cucumovirus, fabavirus, furovirus, hordeivirus, ilarvirus, luteovirus, potexvirus, potyvirus, tobamovirus, tobravirus, tombusvirus, and many others. Other plant viruses exist which have single-stranded antisense (−) RNA (e.g., rhabdoviridae), double-stranded (ds) RNA (e.g., cryptovirus, reoviridae), or ss or ds DNA genomes (e.g., geminivirus and caulimovirus, respectively).

Preferred embodiments of the invention include evolved vectors which are either RNA or DNA viruses. Examples of such embodiments include viruses selected from among: an alfamovirus, a bromovirus, a capillovirus, a carlavirus, a carmovirus, a caulimovirus, a closterovirus, a comovirus, a cryptovirus, a cucumovirus, a dianthovirus, a fabavirus, a fijivirus, a furovirus, a geminivirus, a hordeivirus, a ilarvirus, a luteovirus, a machlovirus, a maize chlorotic dwarf virus, a marafivirus, a necrovirus, a nepovirus, a parsnip yellow fleck virus, a pea enation mosaic virus, a potexvirus, a potyvirus, a reovirus, a rhabdovirus, a sobemovirus, a tenuivirus, a tobamovirus, a tobravirus, a tomato black ring virus, a tomato spotted wilt virus, a tombusvirus, and a tymovirus.

Plant viruses can be engineered as vectors to accomplish a variety of functions. Examples of both DNA and RNA viruses have been used as vectors for gene replacement, gene insertion, epitope presentation and complementation, (see, e.g., Scholthof, Scholthof and Jackson, (1996) “Plant virus gene vectors for transient expression of foreign proteins in plants,” Annu. Rev. of Phytopathol. 34:299-323.) The nucleotide sequences encoding many of these proteins are matters of public knowledge, and accessible through any of a number of databases, e.g. (Genbank: www.ncbi.nlm.nih.gov/genbank/ or EMBL: www.ebi.ac.uk.embl/).

Methods for the transformation of plants and plant cells using sequences derived from plant viruses include the direct transformation techniques described above relating to DNA molecules, see e.g., Jones, ed. (1995) Plant Gene Transfer and Expression Protocols, Humana Press, Totowa, N.J., for a recent compilation. In addition viral sequences can be cloned adjacent T-DNA border sequences and introduced via Agrobacterium mediated transformation, or Agroinfection.

Viral particles comprising the plant virus vectors of the invention can also be introduced by mechanical inoculation using techniques well known in the art, (see e.g., Cunningham and Porter, eds. (1997) Methods in Biotechnology, Vol.3. Recombinant Proteins from Plants: Production and Isolation of Clinically Useful Compounds, for detailed protocols). Briefly, for experimental purposes, young plant leaves are dusted with silicon carbide (carborundum), then inoculated with a solution of viral transcript, or encapsidated virus and gently rubbed. Large scale adaptations for infecting crop plants are also well known in the art, and typically involve mechanical maceration of leaves using a mower or other mechanical implement, followed by localized spraying of viral suspensions, or spraying leaves with a buffered virus/carborundum suspension at high pressure. Any of these above mentioned techniques can be adapted for use with the viral vectors comprising conjoint polynucleotide segments, and/or recombinant concatamers, and are useful for alternative applications depending on the choice of plant virus, and host species, as well as the scale of the specific transformation application.

While the methods of the present invention are suitable for a wide variety of species, including bacteria, fungi, yeast animals and plants, the methods are particularly suited to the improvement of complex phenotypes in plant species. Preferred plants include agronomically and horticulturally important species. Such species include, but are not restricted to members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.), and forest trees (including Pinus, Quercus, Pseutotsuga, Sequoia, Populus, etc.)

Additionally, preferred targets for modification with, e.g., the recombinant concatamers of the invention, as well as those specified above, plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), and the Olyreae, the Pharoideae and many others. As noted, plants in the family Graminae are a particularly preferred target plants for the methods of the invention.

Common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea and nut plants (e.g., walnut, pecan, etc).

The invention described herein furthers the current technology by providing for improved plant phenotypes controlled by various exogenous DNAs as described above. One of skill will recognize that after the exogenous DNA sequence is stably incorporated in transgenic plants and confirmed to be operable, it can be introduced into other plants by sexual crossing. Any of a number of standard breeding techniques can be used, depending upon the species to be crossed.

Libraries of the Invention

Libraries of nucleic acids are collections of cloned DNA fragments that share a common characteristic, e.g., common source (such as an organism, tissue, organ, or cell type), functional characteristic, structural similarity, or are the products of a common process, e.g., diversification (e.g., shuffling) of a pool of DNA sequences as described above. Methods of making libraries of nucleic acids are available and taught, e.g., in Berger, Sambrook and Ausubel, supra. In one embodiment, a library as used in the invention comprises at least 2 nucleic acid sequences. In additional embodiments, the libraries of this invention comprise at least about 2, 5, 10, 100, 1000, or more nucleic acid sequences.

DNA libraries can consist of sequences derived from genomic library. DNA, is extracted from a tissue and either mechanically sheared or enzymatically digested to yield fragments of a desirable size. In the present invention, such fragments are typically between about 25 bp and about 5 kb, e.g., about 15 to about 500, or about 25 to about 200 bp. The fragments are optionally separated by gradient centrifugation from undesired sizes and are ligated in the sense or antisense direction, or a combination thereof, and inserted into a suitable vector, e.g., bacteriophage lambda vectors or plant viral vectors, or artificial chromosomal vector. In the case of viral and phage vectors and the nucleic acids are optionally packaged in vitro.

Alternatively, libraries comprising conjoint genomic fragments are constructed in YAC, or other artificial chromosome vectors. For example, libraries containing large fragments of soybean DNA have been constructed. See, Funke and Kolchinsky (1994) CRC Press, Boca Raton, FL, pp. 125-308 1994; Marek and Shoemaker (1996) Soybean Genet Newsl 23:126-129 1996; Danish et al. (1997) Soybean Genet Newsl 24:196-198. See also, Ausubel, chapter 13 for a description of procedures for making YAC libraries.

Alternatively, libraries can be collections of cDNA molecules corresponding to cellular RNA molecules. Such cDNA libraries, e.g., expression libraries, can be designed to produce either sense or antisense transcripts depending on the orientation of the insert cDNA with respect to the initiation of transcription by a promoter incorporated into the vector. Libraries consisting of cDNA molecules can include DNAs corresponding to predominantly full length or partial RNA transcripts. In one preferred embodiment, inverted cDNAs corresponding to partial transcripts of approximately 15 to about 150, or between 50 to about 100 bp in length are joined end-to-end to produce a library of conjoint polynucleotide segments. These conjoint polynucleotide segments, or a selected subset thereof, can be artificially evolved using a variety of diversification procedures, e.g., DNA shuffling and other mutagenesis techniques, as described herein to produce a library of recombinant concatamers, selected members of which exert desired effects on a complex phenotype.

Kits

The present invention also provides a kit or system for performing one or more of the methods described herein. The kit or system can optionally include a set of instructions for practicing one or more of the methods described herein; one or more assay components that can include at least one recombinant, isolated and/or artificially evolved polynucleotide sequence, nucleic acid, or episomal vector, or at least one cell that includes one or more such sequence or vector, or both; and a container for packaging the set of instructions and components.

In a further aspect, the present invention provides for the use of any component or kit herein, for the practice of any method or assay herein, and/or for the use of any apparatus or kit to practice any assay or method herein.

### EXAMPLES

### Example 1

### Identification and Optimization of Mutliple Elements of a Metabolic Pathway

Despite the large number of proteins, including enzymes, carrier proteins, and transcription factors, involved in determining plant oil composition, the methods of the invention provide a means of rapidly exploring oil “phenotype space.” The following example illustrates how the methods of the invention can be utilized to identify and optimize multiple elements of one or more metabolic pathway involved in the synthesis of seed oil, e.g., in the soybean, Glycine max. Numerous known, and as yet unknown, genes and gene products function to determine the composition and quantity of oil produced and stored in the soybean. Each of these is subject to a variety of environmental and developmental regulatory controls, which are, by-and-large, independently regulated. In order to effect a concerted and desired alteration in the oil phenotype, these many contributory factors must be altered in a coordinated manner.

For example, manipulation of known oil production genes by the methods of the invention can be used to identify important components of the oil production pathway determining composition and quantity. As illustrated schematically in FIG. 1 antisense elements (102) of approximately 50 bp are synthesized corresponding to known oil production related genes (101), e.g., genes encoding enzymes such as stearoyl acyl carrier protein (stearoyl-ACP) desaturases, thioesterases, sn-2 acytransferases, omega 3 fatty acid desaturases, 3-ketoacyl-acyl carrier protein synthases, beta-ketoacyl-CoA synthases, and the like. Additional details and substrates relating to oil synthetic and regulatory proteins are found, e.g., in WO 00/61740 “Modified Lipid Production” by Yuan et al. Alternatively, sequence corresponding to ESTs derived from oil producing organs such as seeds can be used. Similarly, cDNAs corresponding to RNAs expressed in oil producing organs, can provide the sequences for the antisense oligonucleotides.

Typically, multiple, e.g., 3 or 4, antisense elements corresponding to each gene are synthesized. The synthetic oligonucleotides are enzymatically or chemically linked, optionally following synthesis and annealing of the complementary strand. The oligonucleotides are preferably designed to have unique overlapping ends to insure that ligation is directional (i.e., antisense to antisense) and, optionally, in a predetermined order. Duplex DNA corresponding to single stranded joined oligonucleotides, can, where necessary, be synthesized by, e.g., PCR, or other template dependent polymerase reaction to produce conjoint polynucleotide segments. The double stranded conjoint polynucleotide segments are then operably linked to a strong promoter, and optionally, ligated into a vector (FIG. 1, 103), e.g., a plant virus as described above,. Typically, approximately 20 antisense elements of approximately 50 bp each are ligated together in a single viral vector, resulting in an insert size of approximately 1 kb. This length of exogenous DNA is readily accepted by many viral vectors without disrupting essential replication or packaging functions. As shown in FIG. 2, with hundreds of potential targets (202), different combinations of antisense elements are included in a population of vectors (203) to explore the many possibilities for controlling oil synthesis. If an RNA virus is selected, the vector is typically produced in DNA form to simplify construction and manipulation, and then infectious transcripts comprising the joined antisense elements are produced and used to infect suitable host plants.

Following infection (see, FIG. 3), the joined antisense elements (303) are expressed under the regulatory control of a viral, or other strong promoter to produce mRNAs (304). The transformed plants are then screened for alterations in oil production, e.g., by gas chromatography, produced by modulation of endogenous genetic elements (301) by the antisense elements. Virus is recovered from plants exhibiting desired alterations in oil production, and optionally, cDNA corresponding to the viral vector is reverse transcribed.

The conjoint polynucleotide segments are diversified using any of the described mutagenesis or recombination techniques to produce a library of recombinant concatamers. The library of recombinant concatamers is then transfected into host plants and screened to identify those recombinant concatamers that confer a desired alteration in oil composition and/or quantity. Again, the vectors are recovered. After one or more rounds of diversification and screening, vectors that confer the desired alteration in phenotype are recovered, and the elements used singly or in combination to identify and/or isolate the genes involved in achieving the desired alteration in oil production.

Alternatively, rather than joining the elements on a single episomal vector, the synthetic oligonucleotides corresponding to individual genes are manipulated, e.g., introduced, expressed, diversified, screened, etc., in various combinations (subsets) of separate episomes. This variation, similarly, permits the identification of combinations of elements that favorably affect the phenotype of interest.

As shown in FIG. 4, the individual components (402) of the recombinant concatamer (401) can themselves be used to isolate additional family members (403) related to the identified genes, and singly or in combination, can be subjected to the diversification, e.g., recombination and recursive recombination in vitro or in vivo, and selection procedures as described above to derive optimized variants of the individual genes (404) contributing to the complex phenotype. Regardless of whether one, or a few, major control genes, or several biosynthetic enzymes, or a combination of control genes and biosynthetic enzymes are involved, they can be identified and then improved by the methods described herein.

### Example 2

### Identification and Optimization of Multiple Elements of a Genetic Pathway

In multicellular eukaryotes, differentiation of distinct cell types, each with a unique set of expressed proteins, is the result of complex genetic pathways, often regulated by a combination of environmental influences and cellular factors. The ability to transdifferentiate a desired cell type, or subtype, from, e.g., a cell line that is easily grown in culture is of great utility in a vast variety of therapeutic and experimental applications. Cellular factors include a wide variety of nuclear and cytoplasmic components, including nuclear and cytoplasmic proteins, RNAs riboproteins, and the like. The interactions between these cellular factors, between various cellular factors and the environment, and between the various cellular factors and the chromosomal (and nonchromosomal) genetic constitution of the cell, define the genetic program that determines the differentiation pathway. The present invention provides a means of exploring and identifying which of these many factors determine, execute and maintain cell fate decisions.

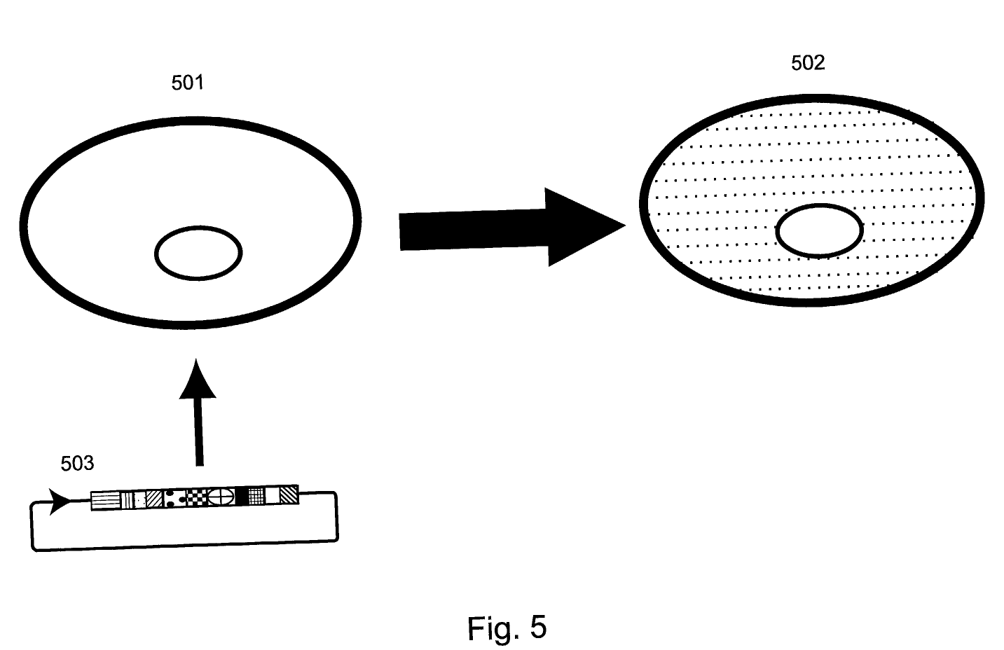

For example, cytoplasmic RNAs, themselves encoding, e.g., cytoplasmic and/or nuclear proteins, can be used as the template to produce cDNA libraries. As described above, and schematically illustrated in FIG. 5, antisense elements corresponding to members of the cDNA library can be joined together as conjoint polynucleotide segments under the regulatory control of a single strong constitutive promoter (503). Alternatively, subsets of “minigenes” corresponding to members of a cDNA library can be joined together under independent constitutive promoters. Overlapping subsets of elements, whether antisense or minigene elements, making up conjoint polynucleotide segments can be transfected into a host cell line of a first cell type (501), e.g., an easily grown or undifferentiated cell type. The effect on differentiation, or trans-differentiation, to a second cell type (502) is then evaluated by any available assay, e.g., visual assessment of morphology, biochemical characterization, genetic characterization, etc.).

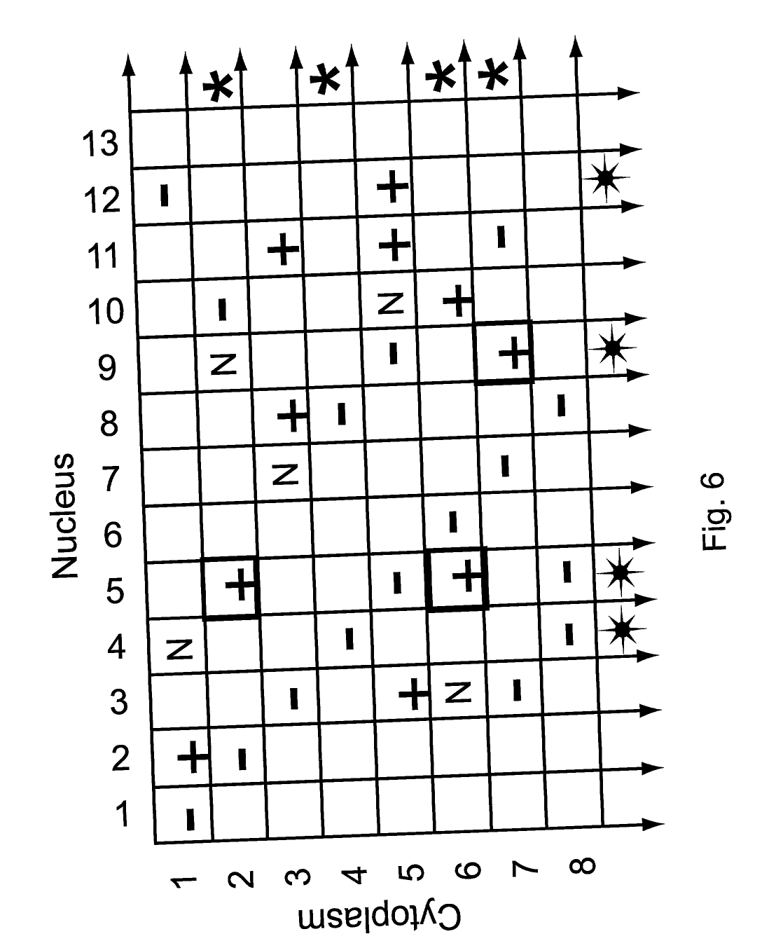

By transfecting multiple cell lines, of differing origins, with duplicate library subsets, a matrix (FIG. 6) can be developed which defines unique subsets of conjoint polynucleotide segments, (comprising sets of cellular cDNAs) capable of effecting trans-differentiation to specified cellular phenotypes, e.g., as evaluated by morphology, cell surface marker or target gene expression profile. Vectors comprising conjoint polynucleotide segments can then be recovered and genes corresponding to the constituent elements isolated and optimized according to the procedures described above for diversification and screening.

### Example 3

### Identification and Optimization of Peptide Modulators of Cellular Targets

Protein or peptide modulators can be used effectively to alter (modify), e.g., inhibit or enhance, the activity of cellular targets. Such cellular targets include a wide variety of intracellular, extracellular and cell-surface molecules, such as enzymes, receptors, hormones, transcription factors, etc. The following example describes the identification and optimization of peptide modulators of enzyme activity, although it will readily be understood that these methods can be adapted to essentially any target or class of targets. Essentially any enzyme for which an activity assay exists or can be developed is a suitable target. For example, proteases, lipases, esterases, hydrolases, and amylases, among many others. Numerous examples of specific enzymes and enzyme classes that provide favorable targets are found in, e.g., PCT/US01/06775 “Single Stranded Nucleic Acid Template-Mediated Recombination and Nucleic Acid Fragment Isolation” by Affholter, as well as in a variety of the references cited herein. Exemplary methods for assaying enzyme activity are found, e.g., Manchenko, G. P., Handbook of Detection of Enzymes on Electrophoretic Gels (CRC Press, Boca Raton, Fla., 1994) and references cited therein. Numerous calorimetric, fluorometric, photospectrometric, chromatographic, electrophoretic, immunologic and other methods are known in the art for assaying specific enzymes and classes of enzymes. Exemplary procedures can be found, e.g., in various Volumes of Methods in Enzymology, e.g., Volumes 1-6 “Preparation and Assay of Enzymes,” Colowick and Kaplan (eds.) Academic Press; Enzyme Assays: A Practical Approach, Eisenthal and Danson (Eds.) Oxford University Press, Oxford; Enzyme Immunoassays: From Concept to Product Development, Deshpande, Chapman and Hall, New York; Enzymology Labfax Engel, Academic Press, Inc., San Diego, as well as in numerous other useful references as found, e.g., in the Molecular Probes Catalogue (2001) http://www.probes.com, Sigma Catalogue (2000), etc.

Novel Peptide modulators, e.g., peptide inhibitors, of an enzyme of interest, e.g., a protease, can be rapidly identified and optimized for one or more property, according to the following procedures. First, a library of polynucleotide segments, e.g., oligonucleotides, encoding potential peptide inhibitors is assembled by pre-selecting a subset of sequences with a desired characteristic from a large and diverse library of nucleic acids. As described above, numerous approaches are available for pre-selecting polynucleotides and/or their encoded products, including polynucleotides encoding peptide or polypeptides with properties of interest. In one particularly favorable approach, a library of short, e.g., about 5 to about 50 amino acid, or about 5 to about 100 amino acid peptides are expressed in the context of a bacterial display fusion protein. For example, polynucleotide segments encoding variable peptide moieties corresponding to the library of peptides to be screened, e.g., random N-mers, partially randomized peptides, peptides chosen by design based on structural or sequence criteria, or any combination of the above, are ligated into a cloning (or multicloning) site engineered into the bacterial cell surface protein OmpA. The fusions are expressed in E. coli, and the fusion polypeptide incorporating the variable peptide moiety is displayed on the bacterial cell surface. Those variable peptide moieties that are able to bind, either in a substrate binding site (i.e., a catalytic site of the enzyme), allosterically, or otherwise, are detected and recovered by staining the cells with a fluorescently labeled protease of choice. The chosen protease can be a naturally occurring isolated or cloned protease, or an artificial model protease incorporating features representative of a subset of proteases, e.g., papain-like cysteine proteases. Indeed, at this juncture, the preferred or “best” enzymatic target for achieving a desired phenotype, need not even be known or isolated.

The cells stained with (i.e., capable of binding to) the labeled enzyme are then detected by Flow Cytometry and sorted, i.e., by Fluorescence Activated Cell Sorting (FACS). For additional details of exemplary procedures adaptable to the present methods, see, e.g., Daugherty et al. (200) “Flow Cytometric Screening of cell-based libraries” J. Immun. Methods 243:211-227; Olsen et al. (200) “High-throughput screening of enzyme libraries” Current Opinion in Biotech. 11:331-337; Olsen et al. (2000) “Function-based isolation of novel enzymes from a large library” Nature Biotechnology 18: 1071-1074; Daugherty ((1999) “Development of an optimized expression system for the screening of antibody libraries displayed on the Escherichia coli surface” Protein Engin 12: 613-621; and Daugherty et al. (1998) “Antibody affinity maturation using bacterial surface display” Protein Engineering 11: 825-832. Alternatively, such methods as ribosomal display, phage display, or yeast display can be utilized to evaluate binding of peptides to a target of interest. Polynucleotide segments encoding the selected variable peptide moieties are then recovered, e.g., by standard cloning procedures or by the polymerase chain reaction.

Following identification of a library of pre-selected candidates, the peptides can, if so desired, at this point be assayed for their ability to modulate, e.g., inhibit activity of a target enzyme.

To optimize the modulatory properties of the pre-selected candidate peptides, the polynucleotides segments encoding the peptides are assembled into conjoint polynucleotide segments encoding a “multipeptide” made up of multiple individual candidate peptides. The components of a single multipeptide can be either the same or different peptides, and can exhibit the same or different activities in a screening assay. The peptides can be assembled in a direct end-to-end arrangement, or they can be assembled such that the individual peptides are separated by a linker sequence, e.g., a linker subject to proteolytic or other cleavage, and/or incorporating a restriction enzyme recognition sequence. The only requirement is that the individual polynucleotide segments be assembled in such a manner that the reading (coding) frame of the individual peptide segments is maintained. The conjoint polynucleotide segments encoding multipeptides are operably linked, i.e., cloned under the transcriptional control, of appropriate regulatory sequences, e.g., promoter, enhancer sequences, chosen to direct transcription in a recipient cell type of interest. Typically, the conjoint polynucleotide sequences are cloned into a vector to facilitate subsequent manipulations, e.g., introduction into the recipient cell, recovery following selection.

The conjoint polynucleotide segments are then introduced and expressed in a recipient cell of choice, e.g., selected based on a target or phenotype of interest. Translation of the multipeptide overcomes the difficulty of obtaining significant expression of small peptides, often encountered when attempting to express small peptides individually within cells. By linking the individual peptide sequences together, significantly higher concentrations of the peptides can be obtained. If desired, cleavage within the linkers can be used to liberate the individual peptide components. The ability of the multipeptide components to modulate activity of the target protease is then evaluated, by standard methods, as described above. In some cases, different peptides, each capable of binding to or modulating a particular class of enzyme, are joined together, providing the basis for broad spectrum modulation of a group of related enzymes.

At this point, the conjoint polynucleotide segments can be diversified, e.g., recombined and/or mutated, to generate a large library of recombinant concatamers encoding multipeptides, the components of which are peptide modulators. In some cases, joining of polynucleotide segments encoding peptides via a common linker sequence provides additional regions of sequence similarity increasing recombination between units with low sequence similarity. The diversified library is then selected or screened, as discussed above, to identify recombinant concatamers that have improved, e.g., optimized, modulatory activities. According to these methods, modulators can be developed regardless of the knowledge of the specific target enzyme. For example, when little or no information is available regarding the “best” target for a phenotype of interest, a library of peptide modules consisting of pre-selected peptides with binding activity for a general class of targets, can be assembled, e.g., via linkers, can be randomly assembled in various combinations and diversified. The resulting library of recombinant or chimeric peptides can then be screened in the cell or organism of interest to obtain the most effective subset of peptide modulators. Subsequent rounds of diversification, e.g., by recombination and/or mutation, can then be used to further optimize the effectiveness of the components of the multipeptide against the specific cellular target of interest.

Although the foregoing has been described in terms of in vivo assay systems, in vitro transcription and/or translation systems can also be employed, including, e.g., ribosomal display methods as described above, and in, e.g., PCT/US01/01056 “Integrated Systems and Methods for Diversity Generation and Screening” by Bass et al.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for controlling a phenotype, the method comprising:
(i) providing a population of conjoint polynucleotide segments, wherein one or more conjoint polynucleotide segments comprise, encode or modulate a phenotype;
(ii) recombining or mutating the population of conjoint polynucleotide segments to produce a library of recombinant concatamers;
(iii) introducing the library of recombinant concatamers into a population of recipient cells or intracellular organelles; and,
(iv) identifying at least one recipient cell, intracellular organelle, or organism comprising a recipient cell, with a desired phenotype, thereby controlling the phenotype.

2. The method of claim 1, comprising controlling a complex phenotype by providing a population of conjoint polynucleotide segments, wherein one or more conjoint polynucleotide segments comprise, encode or modulate at least one element of a multigenic phenotype.

3. The method of claim 1, comprising controlling a complex phenotype by providing a population of conjoint polynucleotide segments, wherein one or more conjoint polynucleotide segments comprise, encode or modulate at least two elements of a multigenic phenotype.

4. The method of claim 1, comprising pre-selecting at least one polynucleotide sequence comprising conjoint polynucleotide segments by (a) introducing a population of random or selected conjoint polynucleotide segments into a plurality of recipient cells, (b) selecting at least one recipient cell, intracellular organelle, or organism comprising a recipient cell, with a desired phenotype, and (c) recovering at least one polynucleotide sequence comprising conjoint polynucleotide segments from the at least one recipient cell with a desired phenotype, thereby pre-selecting at least one polynucleotide sequence comprising conjoint polynucleotide segments.

5. The method of claim 1, comprising providing a population of conjoint polynucleotide segments comprising random polynucleotide segments.

6. The method of claim 1, comprising providing a population of conjoint polynucleotide segments comprising pre-selected polynucleotide segments.

7. The method of claim 6, wherein the polynucleotide segments are pre-selected for one or more encoded activities.

8. The method of claim 6, comprising identifying the pre-selected polynucleotide segments by computational analysis of at least one genomic or expressed sequence.

9. The method of claim 6, comprising identifying the pre-selected polynucleotide segments by expression analysis using at least one cDNA or oligonucleotide array.

10. The method of claim 6, comprising identifying the pre-selected polynucleotide segments by metabolic modeling and flux analysis.

11. The method of claim 6, comprising identifying the pre-selected polynucleotide segments by screening or selecting encoded peptides.

12. The method of claim 6, comprising identifying the pre-selected polynucleotide segments by Flow Cytometry.

13. The method of claim 6, comprising identifying the pre-selected polynucleotide segments by yeast two-hybrid analysis.

14. The method of claim 6, which pre-selected polynucleotide segments comprise one or more elements of a single metabolic or genetic pathway.

15. The method of claim 6, which pre-selected polynucleotide segments comprise elements of multiple metabolic or genetic pathways.

16. The method of claim 1, wherein the phenotype is regulated by at least one epigenetic mechanism.

17. The method of claim 16, wherein the epigenetic mechanism is selected from: chromatin silencing, methylation, maternal effects, regulation by cytoplasmic factors, antisense suppression, sense suppression, cosuppression, promoter alteration, homology-dependent mechanisms, aminoacylation, post-transcriptional gene silencing, and DNA recombination.

18. The method of claim 17, wherein the post-transcriptional gene silencing comprises post-translational silencing by a dominant negative inhibitor, a transdominant inhibitor, or a peptide inhibitor.

19. The method of claim 1, wherein the conjoint polynucleotide segments comprise one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

20. The method of claim 1, wherein the conjoint polynucleotide segments further comprise a vector.

21. The method of claim 20, wherein the vector comprises an episomal vector.

22. The method of claim 21, which vector is a plasmid, a virus, a provirus, a BAC, a YAC, a transposon, a bacteriophage, or a phagemid.

23. The method of claim 1, comprising introducing a plurality of conjoint polynucleotide segments into a recipient cell.

24. The method of claim 23, wherein the plurality of conjoint polynucleotide segments comprises members of a library of conjoint polynucleotide segments.

25. The method of claim 1, wherein the recipient cell is a bacterium, a yeast, a fungus, a plant cell, or an animal cell.

26. The method of claim 1, wherein the phenotype is selected from among: oil content or composition, fat content or composition, sugar content or composition, starch content or composition, protein content or composition, phytochemical content or composition, nutraceutical content or composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity.

27. The method of claim 1, wherein controlling the phenotype comprises modulating activity of one or more targets.

28. The method of claim 27, wherein the one or more targets comprise one or more enzymes.

29. The method of claim 1, comprising recursively recombining or mutating the population of conjoint polynucleotide segments.

30. The method of claim 1, further comprising performing one or more additional diversity generating techniques prior to the recombining of step (ii).

31. The method of claim 1, wherein the recombinant concatamer is integrated into a chromosome of the recipient cell or organism comprising the recipient cell.

32. The method of claim 1, wherein the recombinant concatamer is integrated into the DNA of an intracellular organelle.

33. The method of claim 1, further comprising recovering at least one recombinant concatamer from the cell, intracellular organelle or organism identified in step (iv), and introducing the recombinant concatamer into a host cell.

34. The method of claim 33, wherein the recombinant concatamer comprises an episomal vector.

35. The method of claim 34, wherein the recombinant concatamer is integrated into a chromosome.

36. The method of claim 33, further comprising regenerating at least one multicellular organism comprising the host cell.

37. The method of claim 33, wherein the host cell is a bacterial cell, a fungal cell, a plant cell or an animal cell.

38. The method of claim 1, further comprising isolating at least one genetic element corresponding to a subsequence of the conjoint polynucleotide segments or recombinant concatamer.

39. The method of claim 38, further comprising:
recombining or mutating the at least one isolated genetic element, thereby producing a library of isolated gene homologues, and selecting at least one gene homologue with a desired property.

40. The method of claim 1, wherein the intracellular organelle comprises a mitochondria or a chloroplast.

41. A method for controlling a phenotype, the method comprising:
(i) providing a library of nucleic acids, which library of nucleic acids comprises a plurality of conjoint polynucleotide segments operably linked to at least one transcription regulatory sequence, which conjoint polynucleotide segments alter expression or activity of at least one component of an endogenous phenotype;
(ii) introducing the library of nucleic acids into a population of recipient cells or intracellular organelles; and
(iii) identifying at least one recipient cell, intracellular organelle or organism comprising a recipient cell, with a desired phenotype, thereby controlling the phenotype.

42. The method of claim 41, comprising controlling a complex phenotype by providing a plurality of conjoint polynucleotide segments that alter expression or activity of at least one component of a multigenic phenotype.

43. The method of claim 41, comprising controlling a complex phenotype by providing a plurality of conjoint polynucleotide segments that alter expression or activity of at least two components of a multigenic phenotype.

44. The method of claim 41, further comprising recombining or mutating a polynucleotide comprising conjoint polynucleotide segments, which polynucleotide confers the desired phenotype on the recipient cell, intracellular organelle or organism comprising the recipient cell identified in step (iii).

45. The method of claim 41, comprising pre-selecting at least one nucleic acid comprising conjoint polynucleotide segments by (a) introducing a population of random or selected conjoint polynucleotide segments into a plurality of recipient cells, (b) selecting at least one recipient cell, intracellular organelle, or organism comprising a recipient cell, with a desired phenotype, and (c) recovering at least one nucleic acid comprising conjoint polynucleotide segments from the at least one recipient cell with a desired phenotype, thereby pre-selecting at least one nucleic acid comprising conjoint polynucleotide segments.

46. The method of claim 41, comprising providing a plurality of conjoint polynucleotide segments comprising random polynucleotide segments.

47. The method of claim 41, comprising a plurality of conjoint polynucleotide segments comprising pre-selected polynucleotide segments.

48. The method of claim 47, wherein the polynucleotide segments are pre-selected for one or more encoded activities.

49. The method of claim 47, comprising identifying the pre-selected polynucleotide segments by computational analysis of at least one genomic or expressed sequence.

50. The method of claim 47, comprising identifying the pre-selected polynucleotide segments by expression analysis using at least one cDNA or oligonucleotide array.

51. The method of claim 47, comprising identifying the pre-selected polynucleotide segments by metabolic modeling and flux analysis.

52. The method of claim 47, comprising identifying the pre-selected polynucleotide segments by screening or selecting encoded peptides.

53. The method of claim 47, comprising identifying the pre-selected polynucleotide segments by Flow Cytometry.

54. The method of claim 47, comprising identifying the pre-selected polynucleotide segments by yeast two-hybrid analysis.

55. The method of claim 47, which pre-selected polynucleotide segments comprise one or more elements of a single metabolic or genetic pathway.

56. The method of claim 47, which pre-selected polynucleotide segments comprise elements of multiple metabolic or genetic pathways.

57. The method of claim 41, wherein the phenotype is regulated by at least one epigenetic mechanism.

58. The method of claim 57, wherein the epigenetic mechanism is selected from: chromatin silencing, methylation, maternal effects, regulation by cytoplasmic factors, antisense suppression, sense suppression, cosuppression, promoter alteration, homology-dependent mechanisms, aminoacylation, post-transcriptional gene silencing, and DNA recombination.

59. The method of claim 58, wherein the post-transcriptional gene silencing comprises post-translational silencing by a dominant negative inhibitor, a transdominant inhibitor, or a peptide inhibitor.

60. The method of claim 41, wherein the conjoint polynucleotide segments comprise one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

61. The method of claim 41, wherein the conjoint polynucleotide segments further comprise a vector.

62. The method of claim 61, wherein the vector comprises an episomal vector.

63. The method of claim 62, which vector is a plasmid, a virus, a provirus, a BAC, a YAC, a transposon, a bacteriophage, or a phagemid.

64. The method of claim 41, further comprising recovering at least one nucleic acid comprising conjoint polynucleotide segments from the cell, intracellular organelle or organism identified in step (iii), and introducing the nucleic acid into a host cell.

65. The method of claim 64, wherein the nucleic acid is integrated into a chromosome.

66. The method of claim 64, further comprising regenerating at least one multicellular organism comprising the host cell.

67. The method of claim 64, wherein the host cell is a bacterial cell, a fungal cell, a plant cell or an animal cell.

68. The method of claim 41, wherein the intracellular organelle comprises a mitochondria or a chloroplast.

69. The method of claim 41, wherein the phenotype is selected from among: oil content or composition, fat content or composition, sugar content or composition, starch content or composition, protein content or composition, phytochemical content or composition, nutraceutical content or composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity.

70. The method of claim 41, wherein controlling the phenotype comprises modulating activity of one or more targets.

71. The method of claim 70, wherein the one or more targets comprise one or more enzymes.

72. The method of claim 41, further comprising isolating at least one genetic element corresponding to a subsequence of the conjoint polynucleotide segments or recombinant concatamer.

73. The method of claim 72, further comprising:
recombining or mutating the at least one isolated genetic element, thereby producing a library of isolated gene homologues, and
selecting at least one gene homologue with a desired property.

74. A method for controlling a complex phenotype, the method comprising:
(i) providing a library of nucleic acids, which nucleic acids comprise one or more polynucleotide segments operably linked to at least one transcription regulatory sequence;
(ii) introducing the library of nucleic acids into a population of recipient cells or intracellular organelles, whereby subsets of two or more members of the library, which subsets alter expression or activity of one or more components of a multigenic phenotype, are introduced into a plurality of the recipient cells or organelles; and
(iii) identifying at least one recipient cell, intracellular organelle or organism comprising a recipient cell, with a desired phenotype, thereby controlling the complex phenotype.

75. The method of claim 74, comprising introducing the library of nucleic acids into a population of recipient cells or intracellular organelles, whereby subsets of two or more members of the library, which subsets alter expression or activity of two or more components of a multigenic phenotype, are introduced into a plurality of the recipient cells or organelles.

76. The method of claim 74, comprising providing a library of random polynucleotide segments.

77. The method of claim 74, comprising providing a library of pre-selected polynucleotide segments.

78. The method of claim 77, comprising pre-selecting at least one polynucleotide segment by (a) introducing a population of random or selected polynucleotide segments into a plurality of recipient cells, (b) selecting at least one recipient cell, intracellular organelle, or organism comprising a recipient cell, with a desired phenotype, and (c) recovering one or more polynucleotide segments from the at least one recipient cell with a desired phenotype, thereby pre-selecting one or more polynucleotide segments.

79. The method of claim 77, wherein the polynucleotide segments are pre-selected for one or more encoded activities.

80. The method of claim 77, comprising identifying the pre-selected polynucleotide segments by computational analysis of at least one genomic or expressed sequence.

81. The method of claim 77, comprising identifying the pre-selected polynucleotide segments by expression analysis using at least one cDNA or oligonucleotide array.

82. The method of claim 77, comprising identifying the pre-selected polynucleotide segments by metabolic modeling and flux analysis.

83. The method of claim 77, comprising identifying the pre-selected polynucleotide segments by screening or selecting encoded peptides.

84. The method of claim 77, comprising identifying the pre-selected polynucleotide segments by Flow Cytometry.

85. The method of claim 77, comprising identifying the pre-selected polynucleotide segments by yeast two-hybrid analysis.

86. The method of claim 77, which pre-selected polynucleotide segments comprise one or more elements of a single metabolic or genetic pathway.

87. The method of claim 77, which pre-selected polynucleotide segments comprise elements of multiple metabolic or genetic pathways.

88. The method of claim 74, wherein the phenotype is regulated by at least one epigenetic mechanism.

89. The method of claim 88, wherein the epigenetic mechanism is selected from: chromatin silencing, methylation, maternal effects, regulation by cytoplasmic factors, antisense suppression, sense suppression, cosuppression, promoter alteration, homology-dependent mechanisms, aminoacylation, post-transcriptional gene silencing, and DNA recombination.

90. The method of claim 89, wherein the post-transcriptional gene silencing comprises post-translational silencing by a dominant negative inhibitor, a transdominant inhibitor, or a peptide inhibitor.

91. The method of claim 74, the polynucleotide segments comprising one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

92. The method of claim 74, wherein the nucleic acids further comprise a vector.

93. The method of claim 92, wherein the vector comprises an episomal vector.

94. The method of claim 93, which vector is a plasmid, a virus, a provirus, a BAC, a YAC, a transposon, a bacteriophage, or a phagemid.

95. The method of claim 74, wherein the recipient cells comprise a bacterium, a yeast, a fungus, a plant cell, or an animal cell.

96. The method of claim 74, wherein the phenotype is selected from among: oil content or composition, fat content or composition, sugar content or composition, starch content or composition, protein content or composition, phytochemical content or composition, nutraceutical content or composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity.

97. The method of claim 74, wherein controlling the phenotype comprises modulating activity of one or more targets.

98. The method of claim 97, wherein the one or more targets comprise one or more enzymes.

99. The method of claim 74, further comprising identifying or recovering one or more members of the library from the cells with a desired phenotype identified in step (iii).

100. The method of claim 99, further comprising recombining or mutating the one or more identified or recovered members of the library, thereby producing at least one recombinant polynucleotide segment.

101. The method of claim 100, comprising recursively recombining or mutating the one or more members of the library.

102. The method of claim 100, further comprising introducing the at least one recombinant polynucleotide segment into a host cell.

103. The method of claim 102, wherein the at least one recombinant polynucleotide segment is integrated into a chromosome.

104. The method of claim 102, further comprising regenerating at least one multicellular organism comprising the host cell.

105. The method of claim 102, wherein the host cell is a bacterial cell, a fungal cell, a plant cell or an animal cell.

106. The method of claim 99, further comprising isolating at least one genetic element corresponding to a library member or recombinant polynucleotide segment.

107. The method of claim 106, further comprising:
recombining or mutating the at least one isolated genetic element, thereby producing a library of isolated gene homologues, and
selecting at least one gene homologue with a desired property.

108. The method of claim 74, wherein the intracellular organelle comprises a mitochondria or a chloroplast.

109. A method of modulating activity of one or more targets, the method comprising:
(a) providing a library of polynucleotide segments encoding a plurality of peptides, which peptides are pre-selected for one or more desired properties, which desired properties are the same or different between peptides;
(b) joining the pre-selected polynucleotide segments to generate a population of conjoint polynucleotide segments, which conjoint polynucleotide segments are operably linked to at least one transcription regulatory sequence;
(c) expressing one or more of the conjoint polynucleotide segments in vitro or in vivo, thereby producing one or more multipeptides, which multipeptides comprise a plurality of peptide segments, which segments are optionally joined by a linker sequence;
(d) identifying one or more conjoint polynucleotide segments encoding a multipeptide, which multipeptide comprises at least one peptide capable of modulating activity of one or more targets.

110. The method of claim 109, further comprising:
(e) recombining or mutating the one or more conjoint polynucleotide segments encoding a multipeptide one or more times to produce a library of recombinant concatamers;
(f) expressing one or more recombinant concatamers; and,
(g) identifying at least one recombinant concatamer with a desired property.

111. The method of claim 110, comprising recursively recombining or mutating the one or more conjoint polynucleotide segments.

112. The method of claim 110, comprising performing one or more additional diversity generating techniques prior to recombining the one or more conjoint polynucleotide segments.

113. The method of claim 109, wherein the peptides comprise different amino acid sequences.

114. The method of claim 109, wherein the peptides comprise peptide modulators.

115. The method of claim 109, wherein the peptide modulators comprise peptide inhibitors.

116. The method of claim 109, wherein the peptides modulate an enzyme.

117. The method of claim 109, wherein the polynucleotide segments comprise synthetic oligonucleotides.

118. The method of claim 109, wherein the polynucleotide segments are produced by a polymerase chain reaction (PCR).

119. The method of claim 109, wherein the library comprises random polynucleotide segments.

120. The method of claim 109, wherein the library comprises pre-selected polynucleotide segments.

121. The method of claim 109, comprising pre-selecting the library of polynucleotide segments by (a) introducing a plurality of random, partially randomized or designed polynucleotide segments into a population of recipient cells, (b) selecting one or more recipient cells with a desired phenotype, and (c) recovering at least one polynucleotide segment from the one or more recipient cells with a desired phenotype, thereby pre-selecting the library of polynucleotide segments.

122. The method of claim 121, comprising introducing the plurality polynucleotide segments into a population of recipient cells, wherein the polynucleotide segments are expressed as fusion proteins on the surface of the recipient cell.

123. The method of claim 122, comprising selecting one or more recipient cells expressing a fusion protein that binds to a labeled target.

124. The method of claim 109, wherein the polynucleotide segments are pre-selected for one or more encoded activities.

125. The method of claim 109, comprising identifying the pre-selected polynucleotide segments by computational analysis of at least one genomic or expressed sequence.

126. The method of claim 109, comprising identifying the pre-selected polynucleotide segments by expression analysis using at least one cDNA or oligonucleotide array.

127. The method of claim 109, comprising identifying the pre-selected polynucleotide segments by metabolic modeling and flux analysis.

128. The method of claim 109, comprising identifying the pre-selected polynucleotide segments by screening or selecting encoded peptides.

129. The method of claim 109, comprising identifying the pre-selected polynucleotide segments by phage display, bacterial display, yeast display, or ribosomal display.

130. The method of claim 109, comprising identifying the pre-selected polynucleotide segments by Flow Cytometry.

131. The method of claim 109, comprising identifying the pre-selected polynucleotide segments by yeast two-hybrid analysis.

132. The method of claim 109, comprising providing the library of pre-selected peptides by:
(a) providing a library of nucleic acids encoding fusion polypeptides, which fusion polypeptides are capable of displaying one or more variable peptide moiety in vitro or in vivo;
(b) expressing the fusion polypeptides such that the one or more variable peptide moieties is displayed in vitro or in vivo; and,
(c) identifying a plurality of variable peptide moieties with a desired property, thereby producing a library of pre-selected peptides.

133. The method of claim 109, the polynucleotide segments comprising one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

134. The method of claim 109, wherein the conjoint polynucleotide segments further comprise a vector.

135. The method of claim 134, wherein the vector comprises an episomal vector.

136. The method of claim 135, which vector is a plasmid, a virus, a provirus, a BAC, a YAC, a transposon, a bacteriophage, or a phagemid.

137. The method of claim 109, wherein modulating activity of the one or more targets modifies a phenotype selected from among: oil content or composition, fat content or composition, sugar content or composition, starch content or composition, protein content or composition, phytochemical content or composition, nutraceutical content or composition, yield, time to maturity, growth rate, height at maturity, carbon-fixation rate, salt-tolerance, heat tolerance, cold tolerance, drought tolerance, water-tolerance, heavy metal tolerance, radiation tolerance, resistance to a chemical composition, disease resistance, insect resistance, parasite resistance, color, fluorescence, height, weight, density, toxicity, flavor, sweetness, bitterness, nutritional activity, or therapeutic activity.

138. The method of claim 109, wherein the one or more targets comprise one or more enzymes.

139. The method of claim 138, wherein the one or more targets comprise a class of enzymes.

140. The method of claim 138, wherein the one or more targets comprise at least two different enzymes, which different enzymes are modulated by one or more peptide components of the multipeptide.

141. The method of claim 138, wherein the one or more targets comprise one or more enzymes with multiple functions, which multiple functions are modulated by one or more peptide components of the multipeptide.

142. The method of claim 109, wherein at least one peptide modulates activity by binding to the target.

143. The method of claim 142, wherein the at least one peptide binds to a catalytic site of the target.

144. The method of claim 109, comprising modulating one or more target selected from among intracellular molecules, extracellular molecules and cell-surface molecules.

145. The method of claim 109, wherein modulating activity of a target comprises one or more of: altering target binding to a substrate, altering catalytic activity of a target, altering anabolic activity of a target, altering stability of a target, altering substrate specificity of a target, altering the ability of a target to function in a selected environment.

146. The method of claim 145, comprising modulating a plurality of targets comprising a multiple members of a class of related enzymes.

147. The method of claim 146, wherein the class of related enzymes comprise proteases, lipases, esterases, hydrolases or amylases.

148. The method of claim 109, comprising joining the pre-selected polynucleotide segments by a ligation reaction or by a polymerase chain reaction (PCR).

149. The method of claim 109, wherein the polynucleotide segments encode pre-selected peptides about 5 to about 100 amino acids in length.

150. The method of claim 109, wherein the linker sequence comprises a polynucleotide encoding a peptide subject to proteolytic cleavage.

151. The method of claim 109, comprising expressing the one or more conjoint polynucleotide segments by one or more of in vitro transcription, in vitro translation, or ribosomal display.

152. The method of claim 109, comprising expressing the one or more conjoint polynucleotide segments by introducing a the plurality of conjoint polynucleotide segments into a population of recipient cells.

153. The method of claim 152, wherein the recipient cells comprise a bacterium, a yeast, a fungus, a plant cell, or an animal cell.

154. A method of producing a library of pre-selected peptides, the method comprising:
(a) providing a library of nucleic acids encoding fusion polypeptides, which fusion polypeptides are capable of displaying one or more variable peptide moiety in vitro or in vivo;
(b) expressing the fusion polypeptides such that the one or more variable peptide moieties is displayed in vitro or in vivo; and,
(c) identifying a plurality of variable peptide moieties with a desired property, thereby producing a library of pre-selected peptides.

155. The method of claim 154, wherein the library of pre-selected peptides comprises in excess of about 100 different members.

156. The method of claim 154, wherein the library of pre-selected peptides comprises in excess of about 1000, in excess of about 10,000, in excess of about 100,000 or in excess of about 1,000,000 different members.

157. The method of claim 154, further comprising recovering one or more nucleic acids encoding the pre-selected peptides.

158. The method of claim 154, further comprising joining a plurality of polynucleotide segments encoding the variable peptide moieties, thereby producing one or more conjoint polynucleotide segments encoding a multipeptide.

159. The method of claim 158, recombining or mutating the one or more conjoint polynucleotide segments encoding a multipeptide to produce a library of recombinant concatamers.

160. The method of claim 159, further comprising identifying at least one recombinant concatamer with a desired property.

161. The method of claim 154, comprising displaying the variable peptide moieties by a ribosomal display method, a phage display method, a bacterial display method or a yeast display method.

162. The method of claim 161, comprising displaying the variable peptide moieties as fusions with a bacterial cell surface protein.

163. The method of claim 162, wherein the fusion polypeptide comprises a bacterial OmpA polypeptide.

164. The method of claim 161, comprising detecting one or more variable peptide moieties with a desired property by binding to a labeled target.

165. The method of claim 164, wherein the labeled target comprises an enzyme.

166. The method of claim 164, comprising detecting the one or more variable peptide moieties with a desired property by Flow Cytometry.

167. A library of pre-selected peptide produced by the method of claim 154.

168. A library of nucleic acids, which library of nucleic acids comprises a plurality of conjoint polynucleotide segments operably linked to at least one transcription regulatory sequence, which conjoint polynucleotide segments alter expression of one or more components of an endogenous phenotype.

169. The library of claim 168, wherein the conjoint polynucleotide segments alter expression of multiple components of an endogenous phenotype.

170. The library of claim 168, wherein the endogenous phenotype comprises a multigenic phenotype.

171. The library of claim 168, the polynucleotide segments comprising a plurality of sense or antisense sequences comprising all or part of one or more of a promoter, an enhancer, and a structural gene.

172. The library of claim 171, wherein the structural gene encodes a transcription regulatory factor, an enzyme, a receptor, a hormone, or a signaling peptide or polypeptide.

173. A library comprising the population of conjoint polynucleotide segments of claim 1, 41 or 109.

174. The library of recombinant concatamers of claim 173, the recombinant concatamers comprising one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

175. The library of claim 173, the recombinant concatamers comprising a plurality of sense or antisense sequences comprising all or part of one or more of a promoter, an enhancer, and a structural gene.

176. The library of claim 173, wherein the structural gene encodes a transcription regulatory factor, an enzyme, a receptor, a hormone, or a signaling peptide or polypeptide.

177. The library of recombinant concatamers of claim 1, 41 or 110.

178. The library of recombinant concatamers of claim 178, the recombinant concatamers comprising one or more of: a genomic DNA, a cDNA, a sense-strand DNA, an antisense DNA, a DNA encoding a dominant negative protein variant or a transdominant protein variant, a DNA encoding a peptide modulator, a DNA encoding a 5-100 amino acid peptide, a DNA or RNA decoy, a viral DNA or RNA, an RNA, a sense-strand RNA, an antisense RNA, a tRNA, a ribozyme, an RNP, and a component of the RNA splicing machinery.

179. The library of claim 178, the recombinant concatamers comprising a plurality of sense or antisense sequences comprising all or part of one or more of a promoter, an enhancer, and a structural gene.

180. The library of claim 178, wherein the structural gene encodes a transcription regulatory factor, an enzyme, a receptor, a hormone, or a signaling peptide or polypeptide.

181. The vector of claim 21, 62 and 135.

182. The vector of claim 181, which vector is a plasmid, a virus, a provirus, a BAC, a YAC, a transposon, a bacteriophage, or a phagemid.

183. A cell or an organism comprising a cell produced by the method of claim 1, 41 or 74.

184. The cell of claim 183, wherein the cell comprises a bacterial cell, a fungal cell, a plant cell or an animal cell.

185. The organism of claim 183, wherein the organism is a bacterium, a yeast, a fungus, a plant or an animal.
